(19) United States
(12) Patent Application Publication
(10) Pub. No.: US 2004/0053948 A1
(43) Pub. Date: Mar. 18, 2004

(54) Compounds, compositions and methods

(75) Inventors: Andrew McDonald, San Francisco, CA (US); Gustave Bergnes, Pacifica, CA (US); David J. Morgans JR., Los Altos, CA (US)

Correspondence Address: BEYER WEAVER & THOMAS LLP, P.O. BOX 778, BERKELEY, CA, 94704-0778, US

(73) Assignee: Cytokinetics, Inc.

(21) Appl. No.: 10/434,987

(22) Filed: May 8, 2003

Related U.S. Application Data
(60) Provisional application No. 60/379,922, filed on May 10, 2002.

(51) Int. Cl. A61K 31/517; A61K 31/4709; C07D 417/02; C07D 413/02; C 07D 4 3/02

(52) U.S. Cl. 514/266.2; 514/266.23; 514/309; 544/284; 546/142

(57) ABSTRACT

Quinazolinedione derivatives useful for treating cellular proliferative disorders and disorders associated with Kif15 kinesin activity are described.

### CROSS-REFERENCE To RELATED PATENT APPLICATIONS

This application claims the benefit of U.S. Provisional Patent Application No. 60/379,922, filed May 10, 2002, which is incorporated herein by reference for all purposes.

### FIELD OF THE INVENTION

The present invention relates to quinazolinedione (and phthalimide) derivatives which are inhibitors of the mitotic kinesin Hs Kif15 and are useful in the treatment of cellular proliferative diseases, for example cancer, hyperplasias, restenosis, cardiac hypertrophy, immune disorders, and inflammation.

### BACKGROUND OF THE INVENTION

Among the therapeutic agents used to treat cancer are the taxanes and vinca alkaloids, which act on microtubules. Microtubules are the primary structural element of the mitotic spindle. The mitotic spindle is responsible for distribution of replicate copies of the genome to each of the two daughter cells that result from cell division. It is presumed that disruption of the mitotic spindle by these drugs results in inhibition of cancer cell division, and induction of cancer cell death. However, microtubules form other types of cellular structures, including tracks for intracellular transport in nerve processes. Because these agents do not specifically target mitotic spindles, they have side effects that limit their usefulness.

Improvements in the specificity of agents used to treat cancer is of considerable interest because of the therapeutic benefits which would be realized if the side effects associated with the administration of these agents could be reduced. Traditionally, dramatic improvements in the treatment of cancer are associated with identification of therapeutic agents acting through novel mechanisms. Examples of this include not only the taxanes, but also the camptothecin class of topoisomerase I inhibitors. From both of these perspectives, mitotic kinesins are attractive targets for new anti-cancer agents.

Mitotic kinesins are enzymes essential for assembly and function of the mitotic spindle, but are not generally part of other microtubule structures, such as in nerve processes. Mitotic kinesins play essential roles during all phases of mitosis. These enzymes are “molecular motors” that transform energy released by hydrolysis of ATP into mechanical force which drives the directional movement of cellular cargoes along microtubules. The catalytic domain sufficient for this task is a compact structure of approximately 340 amino acids. During mitosis, kinesins organize microtubules into the bipolar structure that is the mitotic spindle. Kinesins mediate movement of chromosomes along spindle microtubules, as well as structural changes in the mitotic spindle associated with specific phases of mitosis. Experimental perturbation of mitotic kinesin function causes malformation or dysfunction of the mitotic spindle, frequently resulting in cell cycle arrest and cell death.

An important mitotic kinesin which has been identified is Kif15. Mouse Kif15 (Genbank accession numbers AB001432) was originally identified in a PCR-based search for novel murine kinesins (Nakagawa et al. 1997. Proc Natl Acad Sci U S A 94:9654-9). A portion of the MmKif15 cDNA encoding a fragment of the MmKif15 motor domain was cloned and sequenced. In addition, the mRNA expression of MmKif15 in several tissues from 4 week old mice was examined. The discovery of a new human kinesin motor protein, HsKif15, and the polynucleotides encoding it is described in U.S. Pat. No. 6,355,466 and PCT Publication No. WO 01/88118, each of which is incorporated by reference herein for all purposes.

Mitotic kinesins are attractive targets for the discovery and development of novel antimitotic chemotherapeutics. Accordingly, it is an object of the present invention to provide methods, compounds, and compositions useful in the inhibition of HsKif15, a mitotic kinesin.

### SUMMARY OF THE INVENTION

The present invention provides compositions, compounds, and methods that can be used to treat diseases of proliferating cells. The compounds are inhibitors of HsKif15.

In one aspect, the invention relates to methods for treating cellular proliferative diseases and for inhibiting HsKif15. The methods employ compounds or their pharmaceutically acceptable salts chosen from the group consisting of:

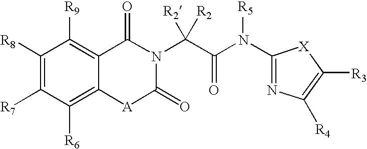

wherein

A is a bond or is —NR₁— wherein R₁ is hydrogen, alkyl, or substituted alkyl;

X is O, S, or —NR₁₂— wherein R₁₂ is hydrogen, alkyl, or substituted alkyl;

R₂ and R₂′ are independently selected from the group consisting of hydrogen, optionally substituted alkyl, optionally substituted aryl, optionally substituted aralkyl, optionally substituted heterocyclyl, and optionally substituted heterocyclylalkyl, or R₂ and R₂′ taken together form an optionally substituted 3- to 7-membered ring;

R₃ is carboxy, alkoxycarbonyl, optionally substituted lower-alkyl or optionally substituted heterocyclyl;

R₄ is hydrogen or optionally substituted lower-alkyl;

R₅ is hydrogen or optionally substituted lower-alkyl; and

R₆, R₇, R₈, and R₉ are independently selected from the group consisting of hydrogen, optionally substituted alkyl, optionally substituted alkoxy, alkoxycarbonyl, halogen, hydroxy, cyano, nitro, amino, alkylamino, dialkylamino, optionally substituted alkylsulfanyl, optionally substituted alkylsulfonyl, optionally substituted alkylsulfonamido, optionally substituted arylsulfonamido, carboxamido, aminocarbonyl, optionally substituted aryl, and optionally substituted heterocyclyl.

Diseases and disorders that respond to therapy with compounds of the invention include cancer, hyperplasia, restenosis, cardiac hypertrophy, immune disorders and inflammation.

In another aspect, the invention relates to compounds useful in inhibiting HsKif15 kinesin. The compounds have the structures shown above.

In an additional aspect, the present invention provides methods of screening for compounds that will bind to HsKif15 kinesin, for example compounds that will displace or compete with the binding of the compounds of the invention. The methods comprise combining a labeled compound of the invention, HsKif15 kinesin, and at least one candidate agent and determining the binding of the candidate bioactive agent to the HsKif15 kinesin.

In a further aspect, the invention provides methods of screening for modulators of HsKif15 kinesin activity. The methods comprise combining a compound of the invention, HsKif15 kinesin, and at least one candidate agent and determining the effect of the candidate bioactive agent on the HsKif15 kinesin activity.

These and other features and advantages of the present invention will be described in more detail below.

### DETAILED DESCRIPTION OF THE EMBODIMENTS

The present invention is directed to a class of novel inhibitors of mitotic kinesins. By inhibiting mitotic kinesins, but not other kinesins (e.g., transport kinesins), specific inhibition of cellular proliferation is accomplished. While not intending to be bound by any theory, the present invention capitalizes on the finding that perturbation of mitotic kinesin function causes malformation or dysfunction of mitotic spindles, frequently resulting in cell cycle arrest and cell death. The methods of inhibiting HsKif15 kinesin comprise contacting an inhibitor of the invention with HsKif15 kinesin. The inhibition can be such that mitosis is disrupted. Meiotic spindles may also be disrupted.

An object of the present invention is to provide inhibitors of mitotic kinesins, in particular HsKif15, for the treatment of disorders associated with cell proliferation. Traditionally, dramatic improvements in the treatment of cancer, one type of cell proliferative disorder, have been associated with identification of therapeutic agents acting through novel mechanisms. Examples of this include not only the taxane class of agents that appear to act on microtubule formation, but also the camptothecin class of topoisomerase I inhibitors. The compounds, compositions and methods described herein can differ in their selectivity and are preferably used to treat diseases of proliferating cells, including, but not limited to cancer, hyperplasias, restenosis, cardiac hypertrophy, immune disorders and inflammation.

Accordingly, the present invention relates to methods employing compounds of the formula:

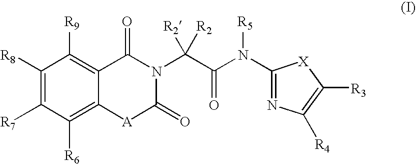

wherein

A is a bond or is —NR₁— wherein R₁ is hydrogen, alkyl, or substituted alkyl;

X is O, S, or —NR₁₂— wherein R₁₂ is hydrogen, alkyl, or substituted alkyl;

R₂ and R₂′ are independently selected from the group consisting of hydrogen, optionally substituted alkyl, optionally substituted aryl, optionally substituted aralkyl, optionally substituted heterocyclyl, and optionally substituted heterocyclylalkyl, or R₂ and R₂′ taken together form an optionally substituted 3- to 7-membered ring;

R₃ is carboxy, alkoxycarbonyl, optionally substituted lower-alkyl, or optionally substituted heterocyclyl;

R₄ is hydrogen or optionally substituted lower-alkyl;

R₅ is hydrogen or optionally substituted lower-alkyl; and

R₆, R₇, R₈, and R₉ are independently selected from the group consisting of hydrogen, optionally substituted alkyl, optionally substituted alkoxy, alkoxycarbonyl, halogen, hydroxy, cyano, nitro, amino, alkylamino, dialkylamino, optionally substituted alkylsulfanyl, optionally substituted alkylsulfonyl, optionally substituted alkylsulfonamido, optionally substituted arylsulfonamido, carboxamido, aminocarbonyl, optionally substituted aryl, and optionally substituted heterocyclyl.

### DEFINITIONS AND ABBREVIATIONS

The following abbreviations and terms have the indicated meanings throughout:

Ac=acetyl

BNB=4-bromomethyl-3-nitrobenzoic acid

Boc=t-butyloxy carbonyl

Bu=butyl

c-=cyclo

CBZ=carbobenzoxy=benzyloxycarbonyl

CDI=carbonyl diimidazole

DBU=diazabicyclo[5.4.0]undec-7-ene

DCM=dichloromethane=methylene chloride=CH₂Cl₂

DCE=dichloroethane

DEAD=diethyl azodicarboxylate

DIC=diisopropylcarbodiimide

DIEA=N,N-diisopropylethyl amine

DMAP=4-N,N-dimethylaminopyridine

DMF=N,N-dimethylformamide

DMSO=dimethyl sulfoxide

DVB=1,4-divinylbenzene

EEDQ=2-ethoxy-1-ethoxycarbonyl-1,2-dihydroquinoline

Et=ethyl

Fmoc=9-fluorenylmethoxycarbonyl

GC=gas chromatography

HATU=0-(7-Azabenzotriazol-1-yl)-1,1,3,3-tetramethyluronium hexafluorophosphate

HMDS=hexamethyldisilazane

HOAc=acetic acid

HOBt=hydroxybenzotriazole

Me=methyl

mesyl=methanesulfonyl

MTBE=methyl t-butyl ether

NMO=N-methylmorpholine oxide

PEG=polyethylene glycol

Ph=phenyl

PhOH=phenol

PfP=pentafluorophenol

PfPy=pentafluoropyridine

PPTS=pyridinium p-toluenesulfonate

Py=pyridine

PyBroP=bromo-tris-pyrrolidino-phosphonium hexafluorophosphate

RT=room temperature

Sat'd=saturated

s-=secondary

t-=tertiary

TBDMS=t-butyldimethylsilyl

TES=triethylsilane

TFA=trifluoroacetic acid

THF=tetrahydrofuran

TMOF=trimethyl orthoformate

TMS=trimethylsilyl

tosyl=p-toluenesulfonyl

Trt=triphenylmethyl

As used in the present specification, the following words and phrases are generally intended to have the meanings as set forth below, except to the extent that the context in which they are used indicates otherwise.

“Alkoxycarbonyl” refers to —(CO)OR, i.e., an ester.

“Alkyl” is intended to include linear, branched, or cyclic hydrocarbon structures and combinations thereof. Lower-alkyl refers to alkyl groups of from 1 to 5 carbon atoms. Examples of lower-alkyl groups include methyl, ethyl, propyl, isopropyl, butyl, s-and t-butyl and the like. Preferred alkyl groups are those of C₂₀ or below. More preferred alkyl groups are those of C₁₃ or below. Cycloalkyl is a subset of alkyl and includes cyclic hydrocarbon groups of from 3 to 14 carbon atoms. Examples of cycloalkyl groups include c-propyl, c-butyl, c-pentyl, norbornyl, adamantyl and the like. In this application, alkyl refers to alkanyl, alkenyl, and alkynyl residues; it is intended to include cyclohexylmethyl, vinyl, allyl, isoprenyl, propargyl, homopropargyl, and the like. When an alkyl residue having a specific number of carbons is named, all geometric isomers having that number of carbons are intended to be encompassed; thus, for example, “butyl” is meant to include n-butyl, sec-butyl, isobutyl and t-butyl; “propyl” includes n-propyl and isopropyl.

“Alkylene” refers to straight or branched chain divalent radical consisting solely of carbon and hydrogen atoms, containing no unsaturation and having from one to six carbon atoms, e.g., methylene, ethylene, propylene, n-butylene and the like. Alkylene is a subset of alkyl, referring to the same residues as alkyl, but having two points of attachment. Examples of alkylene include ethylene (—CH₂CH₂—), propylene (—CH₂CH₂CH₂—), dimethylpropylene (—CH₂C(CH₃)₂CH₂—) and cyclohexylpropylene (—CH₂CH₂CH(C₆H₁₃)).

“Alkylidene” refers to a straight or branched chain unsaturated divalent radical consisting solely of carbon and hydrogen atoms, having from two to six carbon atoms, e.g., ethylidene, propylidene, n-butylidene, and the like. Alkylidene is a subset of alkyl, referring to the same residues as alkyl, but having two points of attachment. The unsaturation present includes at least one double bond.

“Alkylidyne” refers to a straight or branched chain unsaturated divalent radical consisting solely of carbon and hydrogen atoms having from two to six carbon atoms, e.g., propylid-2-ynyl, n-butylid-1-ynyl, and the like. Alkylidyne is a subset of alkyl, referring to the same residues as alkyl, but having two points of attachment. The unsaturation present includes at least one triple bond.

“Alkoxy” or “alkoxyl” refers to an alkyl group, preferably including from 1 to 8 carbon atoms, of a straight, branched, or cyclic configuration, or a combination thereof, attached to the parent structure through an oxygen (i.e., the group alkyl-O—). Examples include methoxy-, ethoxy-, propoxy-, isopropoxy-, cyclopropyloxy-, cyclohexyloxy- and the like. Lower-alkoxy refers to alkoxy groups containing one to four carbons.

“Substituted alkoxy” refers to the group —O-(substituted alkyl). The substitution on the alkyl group generally contains more than only carbon (as defined by alkoxy). One preferred substituted alkoxy group is “polyalkoxy” or —O-(optionally substituted alkylene)-(optionally substituted alkoxy), and includes groups such as —OCH₂CH₂OCH₃, and glycol ethers such as polyethyleneglycol and —O(CH₂CH₂O)ₓCH₃, where x is an integer of about 2-20, preferably about 2-10, and more preferably about 2-5. Another preferred substituted alkoxy group is hydroxyalkoxy or —OCH₂(CH₂)_{y}OH, where y is an integer of about 1-10, preferably about 1-4.

“Acyl” refers to groups of from 1 to 10 carbon atoms of a straight, branched, cyclic configuration, saturated, unsaturated and aromatic and combinations thereof, attached to the parent structure through a carbonyl functionality. One or more carbons in the acyl residue may be replaced by nitrogen, oxygen or sulfur as long as the point of attachment to the parent remains at the carbonyl. Examples include acetyl, benzoyl, propionyl, isobutyryl, t-butoxycarbonyl, benzyloxycarbonyl and the like. Lower-acyl refers to groups containing one to four carbons.

“α-Amino Acids” refer to naturally occurring and commercially available amino acids and optical isomers thereof. Typical natural and commercially available α-amino acids are glycine, alanine, serine, homoserine, threonine, valine, norvaline, leucine, isoleucine, norleucine, aspartic acid, glutamic acid, lysine, omithine, histidine, arginine, cysteine, homocysteine, methionine, phenylalanine, homophenylalanine, phenylglycine, ortho-tyrosine, meta-tyrosine, para-tyrosine, tryptophan, glutamine, asparagine, proline and hydroxyproline. A “side chain of an α-amino acid” refers to the radical found on the α-carbon of an α-amino acid as defined above, for example, hydrogen (for glycine), methyl (for alanine), benzyl (for phenylalanine), and the like.

“Amino” refers to the group —NH₂. The term “substituted amino” refers to the group —NHR or —NRR where each R is independently selected from the group: optionally substituted alkyl-, optionally substituted alkoxy, optionally substituted amino carbonyl-, optionally substituted aryl-, optionally substituted heteroaryl-, optionally substituted heterocyclyl-, acyl-, alkoxycarbonyl-, sulfanyl-, sulfinyl and sulfonyl-, e.g., diethylamino, methylsulfonylamino, furanyl-oxy-sulfonamino.

“Aminocarbonyl-” refers to the group −NR^{c}COR^{b}, —NR^{c}CO₂R^{a}, or —NR^{c}CONR^{b}R^{c}, where

R^{a} is optionally substituted C₁-C₆ alkyl-, aryl-, heteroaryl-, aryl-C₁-C₄ alkyl-, or heteroaryl-C₁-C₄ alkyl-group;

R^{b} is H or optionally substituted C₁-C₆ alkyl-, aryl-, heteroaryl-, aryl-C₁-C₄ alkyl-, or heteroaryl-C₁-C₄ alkyl- group; and

R^{c} is hydrogen or C₁-C₄ alkyl-; and

where each optionally substituted R^{b} group is independently unsubstituted or substituted with one or more substituents independently selected from C₁-C₄ alkyl-, aryl-, heteroaryl-, aryl-C₁-C₄ alkyl-, heteroaryl-C₁-C₄ alkyl-, C₁-C₄ haloalkyl-, —OC₁-C₄ alkyl-, —OC₁-C₄ alkylphenyl-, —C₁-C₄ alkyl-OH, —OC₁-C₄ haloalkyl-, halogen, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for heteroaryl), —CO₂H, —C(O)OC₁-C₄ alkyl-, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl-, —C(O)C₁-C₄ phenyl-, —C(O)C₁-C₄ haloalkyl-, —OC(O)C₁-C₄ alkyl-, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

“Aryl” and “heteroaryl” mean a 5- or 6-membered aromatic or heteroaromatic ring containing 0 or 1-4 heteroatoms, respectively, selected from O, N, or S; a bicyclic 9- or 10-membered aromatic or heteroaromatic ring system containing 0 or 1-4 (or more) heteroatoms, respectively, selected from O, N, or S; or a tricyclic 12- to 14-membered aromatic or heteroaromatic ring system containing 0 or 1-4 (or more) heteroatoms, respectively, selected from O, N, or S. The aromatic 6- to 14-membered carbocyclic rings include, e.g., phenyl-, naphthyl-, indanyl-, tetralinyl-, and fluorenyl and the 5- to 10-membered aromatic heterocyclic rings include, e.g., imidazolyl-, pyridinyl-, indolyl-, thienyl-, benzopyranonyl-, thiazolyl-, furanyl-, benzimidazolyl-, quinolinyl-, isoquinolinyl-, quinoxalinyl-, pyrimidinyl-, pyrazinyl-, tetrazolyl and pyrazolyl-.

“Aralkyl-” refers to a residue in which an aryl moiety is attached to the parent structure via an alkyl residue. Examples include benzyl-, phenethyl-, phenylvinyl-, phenylallyl and the like. “Heteroaralkyl-” refers to a residue in which a heteroaryl moiety is attached to the parent structure via an alkyl residue. Examples include furanylmethyl-, pyridinylmethyl-, pyrimidinylethyl and the like.

“Carboxyalkyl-” refers to the group -alkyl-COOH.

“Halogen” or “halo” refers to fluorine, chlorine, bromine or iodine. Fluorine, chlorine and bromine are preferred. Dihaloaryl, dihaloalkyl, trihaloaryl etc. refer to aryl and alkyl substituted with a plurality of halogens, but not necessarily a plurality of the same halogen; thus 4-chloro-3-fluorophenyl is within the scope of dihaloaryl.

“Heterocyclic ring” refers to a stable 3- to 15-membered ring radical which consists of carbon atoms and from one to five heteroatoms selected from the group consisting of nitrogen, phosphorus, oxygen and sulfur. For purposes of this invention, the heterocyclic ring radical may be a monocyclic, bicyclic or tricyclic ring system, which may include fused or bridged ring systems, and the nitrogen, phosphorus, carbon or sulfur atoms in the heterocyclic ring radical may be optionally oxidized to various oxidation states. In addition, the nitrogen atom may be optionally quaternized; and the ring radical may be partially or fully saturated or aromatic. Examples of such heterocyclic ring radicals include, but are not limited to, azetidinyl, acridinyl, benzodioxolyl, benzodioxanyl, benzofuranyl, carbazoyl, cinnolinyl, dioxolanyl, indolizinyl, naphthyridinyl, perhydroazepinyl, phenazinyl, phenothiazinyl, phenoxazinyl, phthalazinyl, pteridinyl, purinyl, quinazolinyl, quinoxalinyl, quinolinyl, isoquinolinyl, tetrazoyl, tetrahydroisoquinolyl, piperidinyl, piperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, dihydropyridinyl, tetrahydropyridinyl, pyridinyl, pyrazinyl, pyrimidinyl, pyridazinyl, oxazolyl, oxazolinyl, oxazolidinyl, triazolyl, indanyl, isoxazolyl, isoxazolidinyl, morpholinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzoxazolyl, furyl, tetrahydrofuryl, tetrahydropyranyl, thienyl, benzothieliyl, thiamorpholinyl, thiamorpholinyl sulfoxide, thiamorpholinyl sulfone, dioxaphospholanyl, and oxadiazolyl. “Heterocyclyl” refers to a heterocyclic ring radical as defined above, except that the heterocyclyl ring radical may be attached to the main structure at any heteroatom or carbon atom that results in the creation of a stable structure. Oxazolyl and oxadiazolyl are more particular embodiments.

“Heterocyclylalkyl” refers to a radical of the formula —Rₐ-R_{c} where Rₐ is an alkyl radical as defined herein and R_{c} is a heterocyclyl ring radical as defined herein, for example, (4-methylpiperazin-1-yl)methyl, (morpholin-4-yl)methyl, 2-(oxazolin-2-yl)ethyl, and the like.

“Optional” or “optionally” means that the subsequently described event or circumstance may or may not occur, and that the description includes instances where said event or circumstance occurs and instances in which it does not. It will be understood by those skilled in the art with respect to any group containing one or more substituents that such groups are not intended to introduce any substitution or substitution patterns that are sterically impractical and/or synthetically non-feasible and/or inherently unstable.

“Oxadiazyl”- refers to a radical which is an isomer of an oxadiazole, e.g. a 1,2,4 or a 1,3,4-oxadiazyl. Compounds of the invention having substituted oxadiazyl substituents, are named by giving the number designation on the oxadiazyl ring of the substitution on the oxadiazyl ring, followed by the numbering system of the particular oxadiazyl. For example, for a 5-substituted-1,2,4-oxadiazyl derivative, the attachment point of the skeleton of the parent compound (that to which the oxadiazyl is attached) is the 3-position carbon (while the substitution is on the 5-carbon).

“Substituted-” alkyl, aryl, heterocyclyl, and oxadiazyl refer respectively to alkyl, aryl, heterocyclyl, and oxadiazyl wherein one or more (up to about 5, preferably up to about 3) hydrogen atoms are replaced by a substituent independently selected from the group: optionally substituted alkyl (e.g., fluoroalkyl), optionally substituted alkoxy, alkylenedioxy (e.g. methylenedioxy), optionally substituted amino (e.g., alkylamino and dialkylamino), optionally substituted amidino, optionally substituted aryl (e.g., phenyl), optionally substituted aralkyl (e.g., benzyl), optionally substituted aryloxy (e.g., phenoxy), optionally substituted aralkyloxy (e.g., benzyloxy), carboxy (—COOH), alkoxycarbony, carboalkoxy (i.e., acyloxy), carboxyalkyl, carboxamido, aminocarbonyl, benzyloxycarbonylamino (CBZ-amino), cyano, carbonyl, halogen, hydroxy, optionally substituted heterocyclylalkyl, optionally substituted heterocyclyl, nitro, sulfanyl, sulfinyl, sulfonyl, and thio.

“Sulfanyl” refers to the groups: —S-(optionally substituted alkyl), —S-(optionally substituted aryl), and —S-(optionally substituted heterocyclyl).

“Sulfinyl” refers to the groups: —S(O)—H, —S(O)-(optionally substituted alkyl), —S(O)-optionally substituted aryl), —S(O)-(optionally substituted amino), and —S(O)-(optionally substituted heterocyclyl).

“Sulfonyl” refers to the groups: —S(O₂)—H, —S(O₂)-(optionally substituted alkyl), —S(O₂)-optionally substituted aryl), —S(O₂)-(optionally substituted heterocyclyl), —S(O₂)-(optionally substituted alkoxy), —S(O₂)-optionally substituted aryloxy), —S(O₂)-optionally substituted amino), and —S(O₂)-(optionally substituted heterocyclyloxy).

“Yield” for each of the reactions described herein is expressed as a percentage of the theoretical yield.

In some embodiments, as will be appreciated by those in the art, two adjacent carbon containing groups on an aromatic system may be fused together to form a ring structure. Again, the fused ring structure may contain heteroatoms and may be substituted with one or more substitution groups “R”. It should additionally be noted that for cycloalkyl (i.e. saturated ring structures), each position may contain two substitution groups, R and R′.

Some of the compounds of the invention may have imino, amino, oxo or hydroxy substituents off aromatic heterocyclic ring systems. For purposes of this disclosure, it is understood that such imino, amino, oxo or hydroxy substituents may exist in their corresponding tautomeric form, i.e., amino, imino, hydroxy or oxo, respectively.

The compounds of the invention, or their pharmaceutically acceptable salts, may have asymmetric carbon atoms, oxidized sulfur atoms or quaternized nitrogen atoms in their structure.

The compounds of the invention and their pharmaceutically acceptable salts may therefore exist as single stereoisomers, racemates, and as mixtures of enantiomers and diastereomers. The compounds may also exist as geometric isomers. All such single stereoisomers, racemates and mixtures thereof, and geometric isomers are intended to be within the scope of this invention.

Methods for the preparation and/or separation and isolation of single stereoisomers from racemic mixtures or non-racemic mixtures of stereoisomers are well known in the art. For example, optically active (R)- and (S)-isomers may be prepared using chiral synthons or chiral reagents, or resolved using conventional techniques. When desired, the R- and S-isomers may be resolved by methods known to those skilled in the art, for example by: formation of diastereoisomeric salts or complexes which may be separated, for example, by crystallization; via formation of diastereoisomeric derivatives which may be separated, for example, by crystallization, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where a desired enantiomer is converted into another chemical entity by one of the separation procedures described herein, a further step may be required to liberate the desired enantiomeric form. Alternatively, a specific enantiomer may be synthesized by asymmetric synthesis using optically active reagents, substrates, catalysts or solvents, or by converting one enantiomer to the other by asymmetric transformation. For a mixture of enantiomers, enriched in a particular enantiomer, the major component enantiomer may be further enriched by recrystallization.

### COMPOUNDS OF THE INVENTION

Considering formula (I), in a particular embodiment, A is —NR₁ wherein R₁ is hydrogen, alkyl, or substituted alkyl, and X is S. In a more particular embodiment, R₁ is hydrogen.

In a particular embodiment, R₂ and R₂′ are independently selected from the group consisting of hydrogen, optionally substituted alkyl, optionally substituted aryl, optionally substituted aralkyl, optionally substituted heterocyclyl, optionally substituted heterocyclylalkyl, or R₂ and R₂′ taken together form an optionally substituted 3- to 7-membered ring. More particularly, R₂′ is hydrogen. In a more particular embodiment, R₂ is phenyl, lower-alkyl or substituted lower-alkyl. Preferably, R₂ is phenyl, methyl, ethyl, i-propyl, n-propyl, i-butyl, s-butyl, or n-propyl. In a most particular embodiment, the stereogenic center to which R₂ and R₂′ are attached is of the S-configuration.

Suitably, R₃ is carboxy, alkoxycarbonyl, optionally substituted lower-alkyl, or optionally substituted heterocyclyl. More suitably, R₃ is alkoxycarbonyl, or optionally substituted oxadiazyl. In a more particular embodiment, R₃ is —(CO)OR₁₀ wherein R₁₀ is lower-alkyl. Yet more particularly, R₁₀ is methyl, ethyl, or propyl. In another more particular embodiment, R₃ is 3-R₁₁-1,2,4-oxadiazyl, 5-R₁₁-1,2,4-oxadiazyl, or 5-R₁₁-1,3,4-oxadiazyl wherein R₁₁ is lower-alkyl.

Suitably, R₄ is hydrogen or optionally substituted lower-alkyl. More suitably, R₄ is lower-alkyl or substituted lower-alkyl. In a most particular embodiment, R₄ is methyl or trifluoromethyl.

In another embodiment, R₃ and R₄, together with the carbons to which they are bound, form an optionally substituted 5-, 6- or 7-membered ring. The ring may be aliphatic or heterocyclyl.

Suitably, R₅ is hydrogen or optionally substituted lower-alkyl. More Suitably, R₅ is hydrogen or methyl.

Suitably, R₆, R₇, R₈, and R₉ are independently selected from the group consisting of hydrogen, optionally substituted alkyl, optionally substituted alkoxy, alkoxycarbonyl, halogen, hydroxy, cyano, nitro, amino, alkylamino, dialkylamino, optionally substituted alkylsulfanyl, optionally substituted alkylsulfonyl, optionally substituted alkylsulfonamido, optionally substituted arylsulfonamido, carboxamido, aminocarbonyl, optionally substituted aryl, and optionally substituted heterocyclyl. More suitably, R₆, R₇, R₈, and R₉ are independently selected from the group consisting of hydrogen, halogen, hydroxy, lower-alkyl, substituted lower-alkyl, and lower-alkoxy.

In a particular subgenus, A is —NR₁ wherein R₁ is hydrogen, alkyl, or lower-alkyl; X is S; R₂′ is hydrogen and R₂ is optionally substituted lower-alkyl; R₃ is alkoxycarbonyl or optionally substituted oxadiazyl; R₄ is hydrogen or optionally substituted lower-alkyl; R₅ is hydrogen or optionally substituted lower-alkyl; and R₆, R₇, R₈, and R₉ are independently selected from the group consisting of hydrogen, halogen, hydroxy, optionally substituted lower-alkyl, and optionally substituted alkoxy.

In view of the foregoing, it will be appreciated that preferred for the compounds, pharmaceutical formulations, methods of manufacture, and use of the present invention are the following combinations (numbered in Roman numerals I-V) and permutations of substituent groups thereof (sub-grouped, respectively, in increasing order of preference):

I. Any of formula (I) where A is —NR₁ wherein R₁ is hydrogen or optionally substituted lower-alkyl, and X is S.

(a) Especially where the stereogenic center to which R₂ and R₂′ is attached is of the S configuration, and particularly where R₂′ is hydrogen.

1. Particularly those where R₂ is phenyl or lower-alkyl

i. Most particularly, where R₂ is isopropyl.

(b) Especially those where R₃ is alkoxycarbonyl or optionally substituted oxadiazyl.

(c) Especially those where R₄ is hydrogen or optionally substituted lower-alkyl.

(d) Especially those where R₅ is hydrogen or optionally substituted lower-alkyl.

(e) Especially those where R₆, R₇, R₈, and R₉ are independently selected from the group consisting of hydrogen, optionally substituted alkyl, optionally substituted alkoxy, alkoxycarbonyl, halogen, hydroxy, cyano, nitro, amino, alkylamino, dialkylamino, optionally substituted alkylsulfanyl, optionally substituted alkylsulfonyl, optionally substituted alkylsulfonamido, optionally substituted arylsulfonamido, carboxamido, aminocarbonyl, optionally substituted aryl, and optionally substituted heterocyclyl.

1. Particularly those where R₆, R₇, R₈, and R₉ are independently selected from the group consisting of hydrogen, halogen, hydroxy, lower-alkyl, substituted lower-alkyl, and lower-alkoxy.

II. Any of formula (I) where the stereogenic center to which R₂ and R₂′ is attached is of the S configuration, and particularly where R₂′ is hydrogen

(a) Especially those where R₂ is phenyl or lower-alkyl

1. Particularly those where R₂ is isopropyl.

(b) Especially those where R₃ is alkoxycarbonyl or optionally substituted oxadiazyl.

(c) Especially those where R₄ is hydrogen or optionally substituted lower-alkyl.

(d) Especially those where R₅ is hydrogen or optionally substituted lower-alkyl.

(e) Especially those where R₆, R₇, R₈, and R₉ are independently selected from the group consisting of hydrogen, optionally substituted alkyl, optionally substituted alkoxy, alkoxycarbonyl, halogen, hydroxy, cyano, nitro, amino, alkylamino, dialkylamino, optionally substituted alkylsulfanyl, optionally substituted alkylsulfonyl, optionally substituted alkylsulfonamido, optionally substituted arylsulfonamido, carboxamido, aminocarbonyl, optionally substituted aryl, and optionally substituted heterocyclyl.

i. Particularly those where R₆, R₇, R₈, and R₉ are independently selected from the group consisting of hydrogen, halogen, hydroxy, lower-alkyl, substituted lower-alkyl, and lower-alkoxy.

III. Any of formula (I) when R₃ is alkoxycarbonyl or optionally substituted oxadiazyl.

(a) Especially when R₃ is —(CO)OR₁₀ wherein R₁₀ is lower-alkyl.

1. Particularly, those where R₁₀ is methyl, ethyl, or propyl.

(b) Especially those where R₃ is 3-R₁₁-1,2,4-oxadiazyl, 5-R₁₁-1,2,4-oxadiazyl, or 5-R₁₁-1,3,4-oxadiazyl wherein R₁₁ is hydrogen or lower-alkyl.

1. Particularly, those where R₁₁ is methyl.

(c) Especially those where R₂ is phenyl or lower-alkyl

1. Particularly those where R₂ is isopropyl.

(d) Especially those where R₄ is hydrogen or optionally substituted lower-alkyl.

1. Particularly those where R₄ is methyl or trifluoromethyl.

(e) Especially those where R₅ is hydrogen or optionally substituted lower-alkyl.

(f) Especially those where R₆, R₇, R₈, and R₉ are independently selected from the group consisting of hydrogen, optionally substituted alkyl, optionally substituted alkoxy, alkoxycarbonyl, halogen, hydroxy, cyano, nitro, amino, alkylamino, dialkylamino, optionally substituted alkylsulfanyl, optionally substituted alkylsulfonyl, optionally substituted alkylsulfonamido, optionally substituted arylsulfonamido, carboxamido, aminocarbonyl, optionally substituted aryl, and optionally substituted heterocyclyl.

1. Particularly those where R₆, R₇, R₈, and R₉ are independently selected from the group consisting of hydrogen, halogen, hydroxy, lower-alkyl, substituted lower-alkyl, and lower-alkoxy.

IV. Any of formula (I) when R₄ is optionally substituted lower-alkyl.

(a) Especially those where R₄ is methyl or trifluoromethyl.

(b) Especially those where R₂ is phenyl or lower-alkyl

1. Particularly where R₂ is isopropyl.

(c) Especially those where R₃ is alkoxycarbonyl or optionally substituted oxadiazyl

(d) Especially those where R₅ is hydrogen or optionally substituted lower-alkyl.

(e) Especially those where R₆, R₇, R₈, and R₉ are independently selected from the group consisting of hydrogen, optionally substituted alkyl, optionally substituted alkoxy, alkoxycarbonyl, halogen, hydroxy, cyano, nitro, amino, alkylamino, dialkylamino, optionally substituted alkylsulfanyl, optionally substituted alkylsulfonyl, optionally substituted alkylsulfonamido, optionally substituted arylsulfonamido, carboxamido, aminocarbonyl, optionally substituted aryl, and optionally substituted heterocyclyl.

1. Particularly those where R₆, R₇, R₈, and R₉ are independently selected from the group consisting of hydrogen, halogen, hydroxy, lower-alkyl, substituted lower-alkyl, and lower-alkoxy.

V. Any of formula (I) where R₈ and R₉ are hydrogen.

(a) Especially those where R₂ is phenyl or lower-alkyl

1. Particularly where R₂ is isopropyl.

(b) Especially those where R₅ is hydrogen or optionally substituted lower-alkyl.

1. Particularly those where R₅ is hydrogen or methyl.

i. Most particularly, those where R₅ is hydrogen.

(c) Especially those where R₆ and R₇ are independently selected from the group consisting of hydrogen, halogen, hydroxy, lower-alkyl, substituted lower-alkyl, and lower-alkoxy.

Particular compounds include the following:

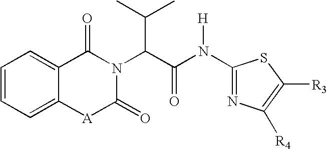

| A | R₃ | R₄ |
|---|---|---|
| Absent | —CO₂Et | —CH₃ |
| —NH | —CO₂Et | —CH₃ |
| —NH | —CH₂OH | —CH₃ |
| —NH | 5-Me-1,3,4-oxadiazole | —CH₃ |
| —NH | 3-Me-1,2,4-oxadiazole | —CH₃ |
| —NH | 5-Me-1,2,4-oxadiazole | —CH₃ |
| —NH | 5-H-1,2,4-oxadiazole | —CH₃ |
| —NH | —CO₂Et | —CF₃ |

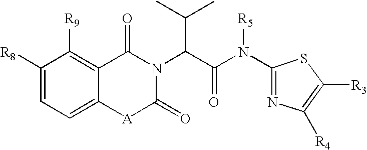

| A | R₃ | R₄ | R₅ | R₈ | R₉ |
|---|---|---|---|---|---|
| —NH | —CO₂Et | —CH₃ | —CH₃ | —H | —H |
| —NH | —CH═CH—CH═CH— | | —H | —H | —H |
| —NH | —N═C(OEt)—CH═CH— | | —H | —H | —H |
| —NCH₃ | —CO₂Et | —CH₃ | —H | —H | —H |
| —NH | —CO₂Et | —CH₃ | —H | —H | —F |
| —NH | —CO₂Et | —CH₃ | —H | —H | —Cl |
| —NH | —CO₂Et | —CH₃ | —H | —Cl | —H |
| —NH | —CO₂Et | —CH₃ | —H | —H | —H |

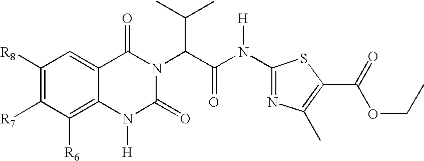

| R₆ | R₇ | R₈ |
|---|---|---|
| —H | —H | —I |
| —H | —H | —F |
| —H | —Cl | —H |
| —H | —CH₃ | —H |
| —H | —F | —H |
| —OCH₃ | —H | —H |
| —Cl | —H | —H |
| —CF₃ | —H | —H |

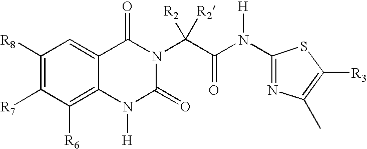

| R₂′ | R₂ | R₃ | R₆ | R₇ | R₈ |
|---|---|---|---|---|---|
| —H | -i-Pr | —CO₂Et | —OCH₃ | —OCH₃ | —OCH₃ |
| —H | —CH₂CH(CH₃)₂ | —CO₂Et | —H | —H | —H |
| —H | —CH(CH₃)CH₂CH₃ | —CO₂Et | —H | —H | —H |
| —CH₃ | —Ph | —CO₂Et | —H | —H | —H |
| —H | —CH₂CH₃ | —CO₂Et | —H | —H | —H |
| —H | —CH₃ | —CO₂Et | —H | —H | —H |
| —H | -i-Pr | —CO₂CH₃ | —H | —H | —H |
| —H | -i-Pr | —CO₂i-Pr | —H | —H | —H |

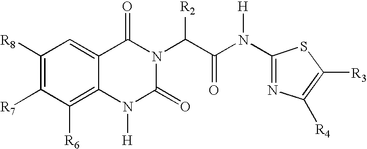

| R₂ | R₃ | R₄ | R₆ | R₇ | R₈ |
|---|---|---|---|---|---|
| -i-Pr | —CO₂n-Pr | —CH₃ | —OCH₃ | —OCH₃ | —OCH₃ |
| -i-Pr | —CO₂Et | —H | —H | —H | —H |
| —CH₂CH₂CH₃ | —CO₂Et | —CH₃ | —H | —H | —H |
| —Ph | —CO₂Et | —CH₃ | —H | —H | —H |

### BRIEF DESCRIPTION OF THE REACTION SCHEMES

Reaction Scheme 1 depicts a synthesis of phthalimide compounds of the invention.

Reaction Scheme 2 depicts a synthesis of quinazolinedione compounds of the invention.

Reaction Scheme 3 depicts another synthesis of quinazolinedione compounds of the invention.

Reaction Scheme 4 depicts a method for alkylating the quinazolinedione 3-nitrogen.

Reaction Scheme 5 depicts synthesis of 3-substituted-1,2,4-oxadiazole derivatives of phthalimide and quinazolinedione compounds of the invention.

Reaction Scheme 6 depicts synthesis of 5-substituted-1,3,4-oxadiazole derivatives of phthalimide and quinazolinedione compounds of the invention.

Reaction Scheme 7 depicts synthesis of 5-substituted-1,2,4-oxadiazole derivatives of phthalimide and quinazolinedione compounds of the invention.

### SYNTHESIS OF COMPOUNDS OF THE INVENTION

The compounds of the invention are synthesized as outlined below, utilizing techniques well known in the art. For example, amines can be condensed with anthranilic acid derivatives and the corresponding amides cyclized using a carbonyl equivalent such as carbonyl diimidazole. Similar approaches are described by Meyer et al. in J. Med. Chem. 2001, 44, 1971-1985; and Negoro et al. in J. Med. Chem. 1998, 41, 4118-4129, both of which are incorporated by reference herein for all purposes.

It is understood that in the following description, combinations of substituents and/or variables on the depicted formulae are permissible only if such combinations result in stable compounds. One skilled in the art would understand that the generic descriptions of the syntheses that follow can have many substitutions of reagents, conditions, and the like without escaping the scope of the invention. The reaction schemes herein are presented for purposes of illustration only and unless otherwise indicated, the following description is directed to the preparation of the compounds of the invention as set forth herein in the summary of the invention as compounds of formula (I).

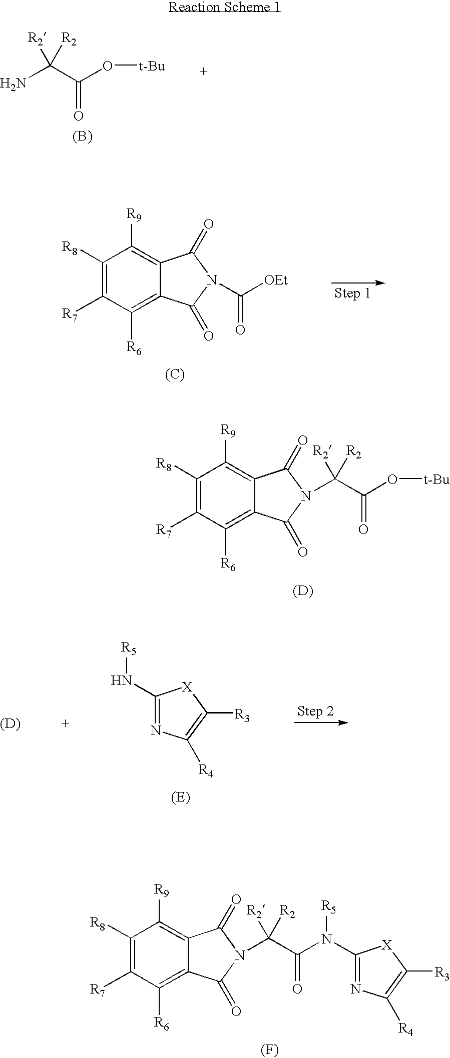

Referring to Reaction Scheme 1, an acid-protected amino acid (B) is converted to the phthalimide derivative (D) via reaction with N-carboethoxyphthalimide (C) (or its equivalent). The acid protecting group is removed (in this case an ester), followed by a peptide coupling (amide bond forming) reaction with amine (E) to make (F). In the case that X═—NR₁₂— wherein R₁₂ is hydrogen, the N must be protected during the coupling reaction and then deprotected. When X═—NR₁₂— wherein R₁₂ is alkyl or substituted alkyl, no protection/deprotection is needed. Further elaboration of (E) or synthesis to make precursors (B) or (E) are understood in the art.

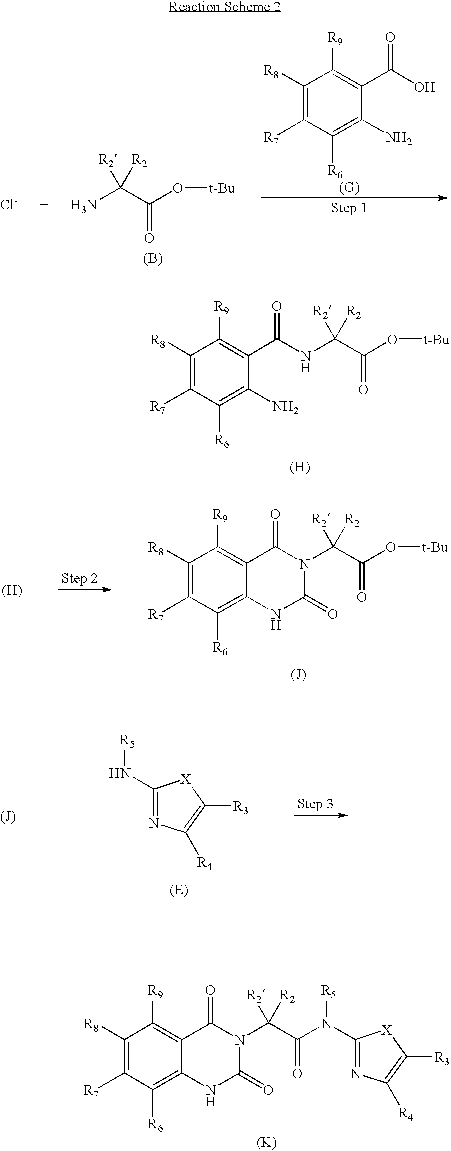

Referring to Reaction Scheme 2, acid-protected amino acid (B) is converted to the amide derivative (H) via a peptide coupling reaction with the corresponding anthranilic acid derivative (G). The quinazolinedione ring structure is formed by closure of the non-aromatic ring via a carbonyl equivalent, such as CDI, to form (J). As before, the acid protecting group is removed (in this case an ester), followed by a peptide coupling (amide bond forming) reaction with amine (E) to make (K). In the case that X═—NR₁₂— wherein R₁₂ is hydrogen, the N must be protected during the coupling reaction and then deprotected. When X═—NR₁₂— wherein R₁₂ is alkyl or substituted alkyl, no protection/deprotection is needed. Further elaboration of (E) or synthesis to make precursors (G) or (E) are understood in the art.

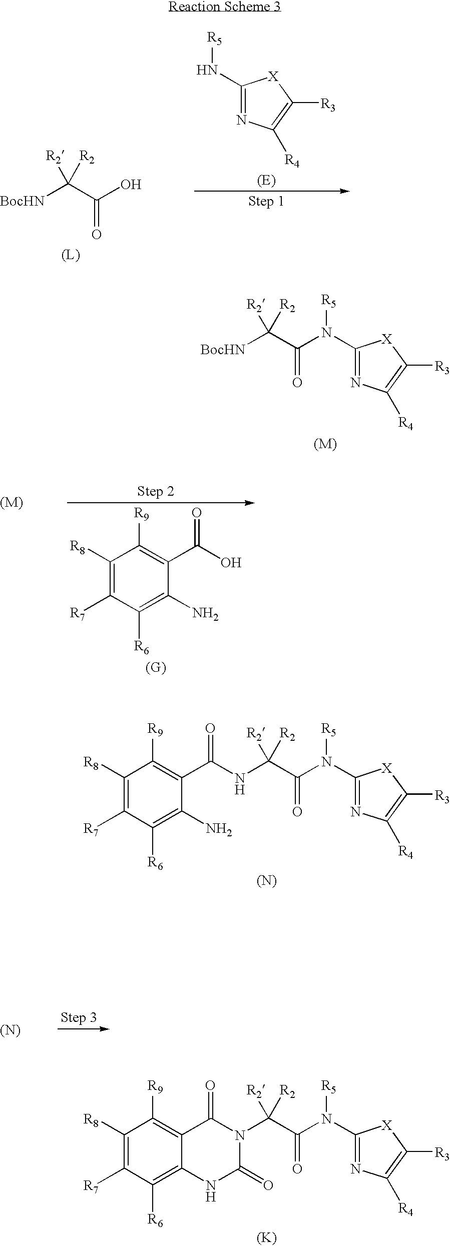

Reaction Scheme 3 shows an alternative for making quinazolinediones of the invention. First, an amine-protected amino acid, (L), is coupled (via a peptide coupling reaction) to (E) to make (M). As before, in the case that X═—NR₁₂— wherein R₁₂ is hydrogen, the N must be protected during the coupling reaction and then deprotected. When X═—NR₁₂— wherein R₁₂ is alkyl or substituted alkyl, no protection/deprotection is needed. The amine-protecting group is removed followed by peptide coupling reaction with (G) to make (N). The quinazolinedione ring structure is formed by closure of the non-aromatic ring via a carbonyl equivalent, such as CDI, to form (K).

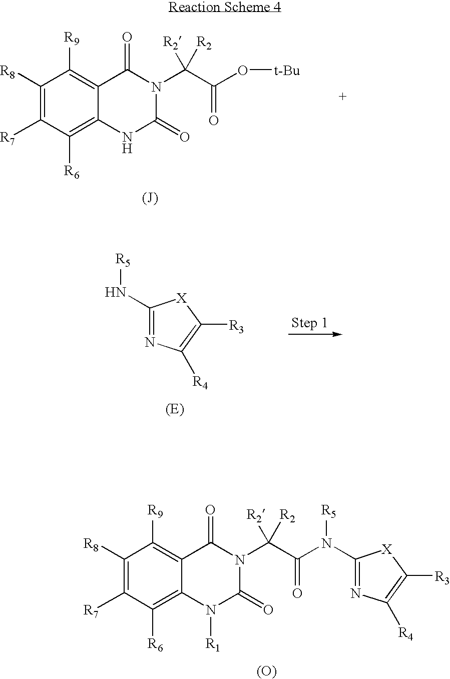

Reaction Scheme 4 shows a general strategy to alkylate the 3-nitrogen of quinazolinediones of the invention (where A is —NR₁ wherein R₁ is optionally substituted lower-alkyl). For example, precursor (J) is deprotonated with a base and then the nitrogen is alkylated with an alkyl halide (or equivalent) containing R₁. Then as previously described, the acid protecting group is removed (in this case an ester), followed by a peptide coupling (amide bond forming) reaction with amine (E) to make (O).

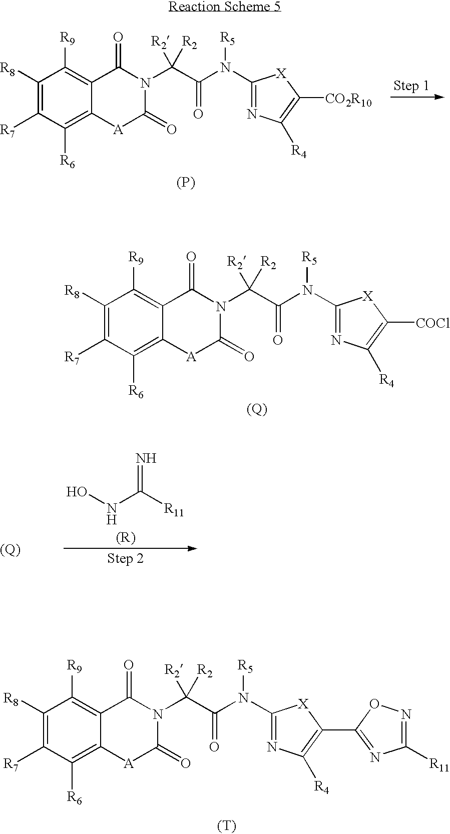

Referring to Reaction Scheme 5, the acid or ester functionality (where R₃═—CO₂R₁₀) of precursor (P) is converted to the acid chloride to give (Q). As before, in the case that X═—NR₁₂— wherein R₁₂ is hydrogen, the N must be protected during the saponification reaction and then deprotected at a later stage. Also, when A=—NR₁ wherein R₁ is hydrogen, the N must be protected during the saponification reaction and then deprotected at a later stage. When X=—NR₁₂— wherein R₁₂ is alkyl or substituted alkyl, or when A=—NR₁ wherein R₁ is alkyl or substituted alkyl no protection/deprotection is needed. Acid chloride (Q) is reacted with hydroxyamidine (R) to give 3-substituted-1,2,4-oxadiazole (T). Either (R) is commercially available or the synthesis of (R) is understood in the art.

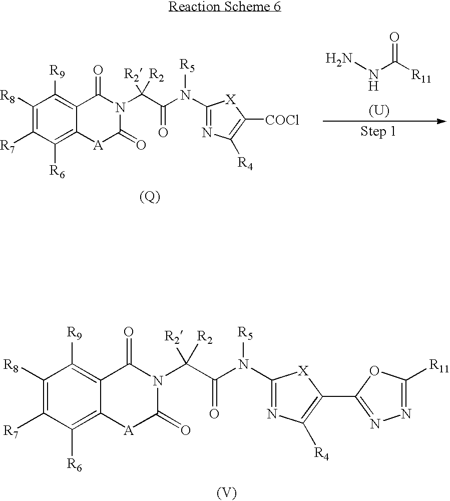

Reaction Scheme 6 shows the synthesis of 1,3,4-oxidiazole derivatives of quinazolinedione or phthalidmide compounds of the invention. Acid chloride (Q) is reacted with N-amino amide (U) to give 1,3,4-oxadiazole (V). Either (U) is commercially available or the synthesis of (U) is understood in the art.

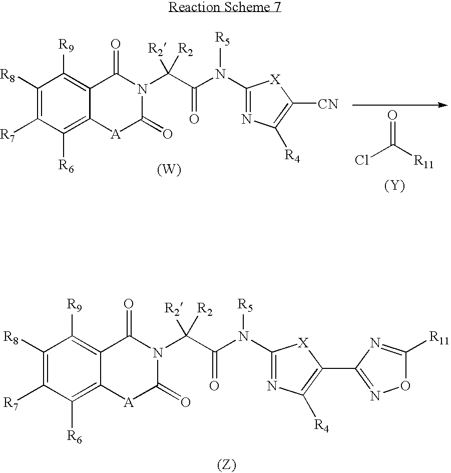

Reaction Scheme 7 shows the synthesis of 5-substituted-1,3,4-oxidiazole derivatives of quinazolinedione and phthalimide compounds of the invention. Nitrile derivative (W) is converted to the corresponding 5-substituted-1,3,4-oxidiazole (Z) by reaction with hydroxylamine followed by acylation with acid chloride (Y), and ring closure of the acylated intermediate (not shown). Either (Y) is commercially available or the synthesis of (Y) is understood in the art.

Utility, Testing and Administration

General Utility

Once made, the compounds of the invention find use in a variety of applications involving alteration of mitosis. As will be appreciated by those skilled in the art, mitosis may be altered in a variety of ways; that is, one can affect mitosis by decreasing the activity of a component in the mitotic pathway. Similar approaches may be used to alter meiosis.

In one embodiment, the compounds of the invention are used to inhibit mitotic spindle formation, thus causing prolonged cell cycle arrest in mitosis. By “inhibit” in this context is meant decreasing or interfering with mitotic spindle formation or causing mitotic spindle dysfunction. By “mitotic spindle formation” herein is meant organization of microtubules into bipolar structures by mitotic kinesins. By “mitotic spindle dysfunction” herein is meant mitotic arrest.

The compounds of the invention are useful to bind to, and/or inhibit the activity of, a mitotic kinesin, Kif15. In one embodiment, the Kif15 is human Kif15, although the compounds may be used to bind to or inhibit the activity of Kif15 kinesins from other organisms. In this context, “inhibit” means either increasing or decreasing spindle pole separation, causing malformation, i.e., splaying, of mitotic spindle poles, or otherwise causing morphological perturbation of the mitotic spindle. Also included within the definition of Kif15 for these purposes are variants and/or fragments of Kif15. See U.S. Pat. No. 6,391,613 and PCT Publication No. WO 01/88118, each of which is hereby incorporated by reference in its entirety.

In another embodiment, the compounds inhibit the mitotic kinesin, Kif15, as well as modulating one or more of the human mitotic kinesins selected from the group consisting of HSET (see, U.S. Pat. No. 6,361,993, which is incorporated herein by reference); MCAK (see, U.S. Pat. No. 6,331,424, which is incorporated herein by reference); CENP-E (see, PCT Publication No. WO 99/13061, which is incorporated herein by reference); Kif4 (see, U.S. Pat. No. 6,440,684, which is incorporated herein by reference); MKLP1 (see, U.S. Pat. No. 6,448,025, which is incorporated herein by reference); KSP (see, U.S. Pat. No. 6,437,115, which is incorporated herein by reference); Kid (see, U.S. Pat. No. 6,387,644, which is incorporated herein by reference); Mpp1, CMKrp, KinI-3 (see, U.S. Pat. No. 6,461,855, which is incorporated herein by reference); Kip3a (see, PCT Publication No. WO 01/96593, which is incorporated herein by reference); Kip3d (see, U.S. Pat. No. 6,492,151, which is incorporated herein by reference); and RabK6.

The compounds of the invention are used to treat cellular proliferation diseases. Such disease states which can be treated by the compounds, compositions and methods provided herein include, but are not limited to, cancer (further discussed below), hyperplasias, restenosis, cardiac hypertrophy, immune disorders, inflammation, and cellular proliferation induced after medical procedures, including, but not limited to, surgery, angioplasty, and the like. Treatment includes inhibiting cellular proliferation. It is appreciated that in some cases the cells may not be in an abnormal state and still require treatment. Thus, in one embodiment, the invention herein includes application to cells or individuals afflicted or subject to impending affliction with any one of these disorders or states.

The compounds, compositions and methods provided herein are particularly deemed useful for the treatment of cancer including solid tumors such as skin, breast, brain, cervical carcinomas, testicular carcinomas, etc. More particularly, cancers that may be treated by the compounds, compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above identified conditions.

Testing

For assay of Kif15-modulating activity, generally either Kif15 or a compound according to the invention is non-diffusably bound to an insoluble support having isolated sample receiving areas (e.g., a microtiter plate, an array, etc.). The insoluble support may be made of any substance to which the sample can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, Teflon™, etc. Microtiter plates and arrays are especially convenient because a large number of assays can be carried out simultaneously, using small amounts of reagents and samples. The particular manner of binding of the sample is not crucial so long as it is compatible with the reagents and overall methods of the invention, maintains the activity of the sample and is nondiffusable. Particular methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the sample, excess unbound material is removed by washing. The sample receiving areas may then be blocked through incubation with bovine serum albumin (BSA), casein or other innocuous protein or other moiety.

The compounds of the invention may be used on their own to inhibit the activity of a mitotic kinesin, particularly Kif15. In one embodiment, a compound of the invention is combined with Kif15 and the activity of Kif15 is assayed. Kinesin (including Kif15) activity is known in the art and includes one or more kinesin activities. Kinesin activities include the ability to affect ATP hydrolysis; microtubule binding; gliding and polymerization/depolymerization (effects on microtubule dynamics); binding to other proteins of the spindle; binding to proteins involved in cell-cycle control; serving as a substrate to other enzymes, such as kinases or proteases; and specific kinesin cellular activities such as spindle pole separation.

Methods of performing motility assays are well known to those of skill in the art. (See e.g., Hall, et al. (1996), Biophys. J., 71: 3467-3476, Turner et al., 1996, AnaL Biochem. 242 (1):20-5; Gittes et al., 1996, Biophys. J. 70(1): 418-29; Shirakawa et al., 1995, J. Exp. BioL 198: 1809-15; Winkelmann et al., 1995, Biophys. J. 68: 2444-53; Winkelmann et al., 1995, Biophys. J. 68: 72S.)

Methods known in the art for determining ATPase hydrolysis activity also can be used. Suitably, solution based assays are utilized. U.S. Pat. No. 6,410,254, hereby incorporated by reference in its entirety, describes such assays. Alternatively, conventional methods are used. For example, Pᵢ release from kinesin can be quantified. In one embodiment, the ATPase hydrolysis activity assay utilizes 0.3 M PCA (perchloric acid) and malachite green reagent (8.27 mM sodium molybdate II, 0.33 mM malachite green oxalate, and 0.8 mM Triton X-100). To perform the assay, 10 μL of the reaction mixture is quenched in 90 μL of cold 0.3 M PCA. Phosphate standards are used so data can be converted to mM inorganic phosphate released. When all reactions and standards have been quenched in PCA, 100 μL of malachite green reagent is added to the relevant wells in e.g., a microtiter plate. The mixture is developed for 10-15 minutes and the plate is read at an absorbance of 650 nm. If phosphate standards were used, absorbance readings can be converted to mM Pᵢ and plotted over time. Additionally, ATPase assays known in the art include the luciferase assay.

ATPase activity of kinesin motor domains also can be used to monitor the effects of agents and are well known to those skilled in the art. In one embodiment ATPase assays of kinesin are performed in the absence of microtubules. In another embodiment, the ATPase assays are performed in the presence of microtubules. Different types of agents can be detected in the above assays. In one embodiment, the effect of a agent is independent of the concentration of microtubules and ATP. In another embodiment, the effect of the agents on kinesin ATPase can be decreased by increasing the concentrations of ATP, microtubules or both. In yet another embodiment, the effect of the agent is increased by increasing concentrations of ATP, microtubules or both.

Compounds that inhibit the biochemical activity of Kif15 in vitro may then be screened in vivo. In vivo screening methods include assays of cell cycle distribution, cell viability, or the presence, morphology, activity, distribution, or number of mitotic spindles. Methods for monitoring cell cycle distribution of a cell population, for example, by flow cytometry, are well known to those skilled in the art, as are methods for determining cell viability. See for example, U.S. Pat. No. 6,437,115, hereby incorporated by reference in its entirety. Microscopic methods for monitoring spindle formation and malformation are well known to those of skill in the art (see, e.g., Whitehead and Rattner (1998), J. Cell Sci. 111:2551-61; Galgio et al, (1996) J. Cell Biol., 135:399-414), each incorporated herein by reference in its entirety.

The compounds of the invention inhibit the Kif15 kinesin. One measure of inhibition is IC₅₀, defined as the concentration of the compound at which the activity of Kif15 is decreased by fifty percent relative to a control. Preferred compounds have IC₅₀'s of less than about 1 mM, with preferred embodiments having IC₅₀'s of less than about 100 μM, with more preferred embodiments having IC₅₀'s of less than about 10 μM, with particularly preferred embodiments having IC₅₀'s of less than about 1 μM, and especially preferred embodiments having IC₅₀'s of less than about 100 nM, and with the most preferred embodiments having IC₅₀'s of less than about 10 nM. Measurement of IC₅₀ is done using an ATPase assay such as described herein.

Another measure of inhibition is Kᵢ. For compounds with IC₅₀'s less than 1 μM, the Kᵢ or K_{d} is defined as the dissociation rate constant for the interaction of the compounds described herein with Kif15. Preferred compounds have Kᵢ's of less than about 100 μM, with preferred embodiments having Kᵢ's of less than about 10 μM, and particularly preferred embodiments having Kᵢ's of less than about 1 μM and especially preferred embodiments having Kᵢ's of less than about 100 nM, and with the most preferred embodiments having Kᵢ's of less than about 10 nM.

The Kᵢ for a compound is determined from the IC₅₀ based on three assumptions and the Michaelis-Menten equation. First, only one compound molecule binds to the enzyme and there is no cooperativity. Second, the concentrations of active enzyme and the compound tested are known (i.e., there are no significant amounts of impurities or inactive forms in the preparations). Third, the enzymatic rate of the enzyme-inhibitor complex is zero. The rate (i.e., compound concentration) data are fitted to the equation:

$$V = {V}_{max} {E}_{0} [ I - \frac{( {E}_{0} + {I}_{0} + K d ) - \sqrt{{( {E}_{0} + {I}_{0} + K d )}^{2} - 4 {E}_{0} {I}_{0}}}{2 {E}_{0}} ]$$

where V is the observed rate, Vₘₐₓ is the rate of the free enzyme, I₀ is the inhibitor concentration, E₀ is the enzyme concentration, and K_{d} is the dissociation constant of the enzyme-inhibitor complex.

Another measure of inhibition is GI₅₀, defined as the concentration of the compound that results in a decrease in the rate of cell growth by fifty percent. Preferred compounds have GI₅₀'s of less than about 1 mM; those having a GI₅₀ of less than about 20 μM are more preferred; those having a GI₅₀ of less than about 10 μM more so; those having a GI₅₀ of less than about 1 μM more so; those having a GI₅₀ of less than about 100 nM more so; and those having a GI₅₀ of less than about 10 nM even more so. Measurement of GI₅₀ is done using a cell proliferation assay such as described herein. Compounds of this class were found to inhibit cell proliferation.

In vitro potency of small molecule inhibitors is determined, for example, by assaying human ovarian cancer cells (SKOV3) for viability following a 72-hour exposure to a 9-point dilution series of compound. Cell viability is determined by measuring the absorbance of formazon, a product formed by the bioreduction of MTS/PMS, a commercially available reagent. Each point on the dose-response curve is calculated as a percent of untreated control cells at 72 hours minus background absorption (complete cell kill).

Anti-proliferative compounds that have been successfully applied in the clinic to treatment of cancer (cancer chemotherapeutics) have GI₅₀'s that vary greatly. For example, in A549 cells, paclitaxel GI₅₀ is 4 nM, doxorubicin is 63 nM, 5-fluorouracil is 1 μM, and hydroxyurea is 500 μM (data provided by National Cancer Institute, Developmental Therapeutic Program, http://dtp.nci.nih.gov/). Therefore, compounds that inhibit cellular proliferation, irrespective of the concentration demonstrating inhibition, may be useful.

To employ the compounds of the invention in a method of screening for compounds that bind to Kif15 kinesin, the Kif15 is bound to a support, and a compound of the invention is added to the assay. Alternatively, the compound of the invention is bound to the support and Kif15 is added. Classes of compounds among which novel binding agents may be sought include specific antibodies, non-natural binding agents identified in screens of chemical libraries, peptide analogs, etc. Of particular interest are screening assays for candidate agents that have a low toxicity for human cells. A wide variety of assays may be used for this purpose, including labeled in vitro protein-protein binding assays, electrophoretic mobility shift assays, immunoassays for protein binding, functional assays (phosphorylation assays, etc.) and the like.

The determination of the binding of the compound of the invention to Kif15 may be done in a number of ways. In a preferred embodiment, the compound is labeled, for example, with a fluorescent or radioactive moiety, and binding is determined directly. For example, this may be done by attaching all or a portion of Kif15 to a solid support, adding a labeled test compound (for example a compound of the invention in which at least one atom has been replaced by a detectable isotope), washing off excess reagent, and determining whether the amount of the label is that present on the solid support.

By “labeled” herein is meant that the compound is either directly or indirectly labeled with a label which provides a detectable signal, e.g., radioisotope, fluorescent tag, enzyme, antibodies, particles such as magnetic particles, chemiluminescent tag, or specific binding molecules, etc. Specific binding molecules include pairs, such as biotin and streptavidin, digoxin and antidigoxin etc. For the specific binding members, the complementary member would normally be labeled with a molecule which provides for detection, in accordance with known procedures, as outlined herein. The label can directly or indirectly provide a detectable signal.

In some embodiments, only one of the components is labeled. For example, the kinesin proteins may be labeled at tyrosine positions using ¹²⁵I, or with fluorophores. Alternatively, more than one component may be labeled with different labels; using ¹²⁵I for the proteins, for example, and a fluorophor for the antimitotic agents.

The compounds of the invention may also be used as competitors to screen for additional drug candidates. “Candidate agent” or “drug candidate” or grammatical equivalents as used herein describe any molecule, e.g., protein, oligopeptide, small organic molecule, polysaccharide, polynucleotide, etc., to be tested for bioactivity. They may be capable of directly or indirectly altering the cellular proliferation phenotype or the expression of a cellular proliferation sequence, including both nucleic acid sequences and protein sequences. In other cases, alteration of cellular proliferation protein binding and/or activity is screened. Screens of this sort may be performed either in the presence or absence of microtubules. In the case where protein binding or activity is screened, suitable embodiments exclude molecules already known to bind to that particular protein, for example, polymer structures such as microtubules, and energy sources such as ATP. Suitable embodiments of assays herein include candidate agents which do not bind the cellular proliferation protein in its endogenous native state termed herein as “exogenous” agents. In another embodiment, exogenous agents further exclude antibodies to Kif15.

Candidate agents can encompass numerous chemical classes, though typically they are organic molecules having a molecular weight of more than 100 and less than about 2,500 daltons. Candidate agents comprise functional groups necessary for structural interaction with proteins, particularly hydrogen bonding and lipophilic binding, and typically include at least an amine, carbonyl-, hydroxyl-, ether, or carboxyl group, and often at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, and/or amidification to produce structural analogs.

Competitive screening assays may be done by combining Kif15 and a drug candidate in a first sample. A second sample comprises a compound of the present invention, Kif15 and a drug candidate. This may be performed in either the presence or absence of microtubules. The binding of the drug candidate is determined for both samples, and a change, or difference in binding between the two samples indicates the presence of a drug candidate capable of binding to Kif15 and potentially inhibiting its activity. That is, if the binding of the drug candidate is different in the second sample relative to the first sample, the drug candidate is capable of binding to Kif15.

In one embodiment, the binding of the candidate agent to Kif15 is determined through the use of competitive binding assays. In this embodiment, the competitor is a binding moiety known to bind to Kif15, such as an antibody, peptide, binding partner, ligand, etc. Under certain circumstances, there may be competitive binding as between the candidate agent and the binding moiety, with the binding moiety displacing the candidate agent.

In one embodiment, the candidate agent is labeled. Either the candidate agent, or the competitor, or both, is added first to Kif15 for a time sufficient to allow binding, if present. Incubations may be performed at any temperature which facilitates optimal activity, typically between 4 and 40° C.

Incubation periods are selected for optimum activity, but may also be optimized to facilitate rapid high throughput screening. Typically between 0.1 and 1 hour will be sufficient. Excess reagent is generally removed or washed away. The second component is then added, and the presence or absence of the labeled component is followed, to indicate binding.

In one embodiment, the competitor is added first, followed by the candidate agent. Displacement of the competitor is an indication the candidate agent is binding to Kif15 and thus is capable of binding to, and potentially inhibiting, the activity of Kif15. In this embodiment, either component can be labeled. Thus, for example, if the competitor is labeled, the presence of label in the wash solution indicates displacement by the agent. Alternatively, if the candidate agent is labeled, the presence of the label on the support indicates displacement.

In an alternative embodiment, the candidate agent is added first, with incubation and washing, followed by the competitor. The absence of binding by the competitor may indicate the candidate agent is bound to Kif15 with a higher affinity. Thus, if the candidate agent is labeled, the presence of the label on the support, coupled with a lack of competitor binding, may indicate the candidate agent is capable of binding to Kif15.

Inhibition is tested by screening for candidate agents capable of inhibiting the activity of Kif15 comprising the steps of combining a candidate agent with Kif15, as above, and determining an alteration in the biological activity of Kif15. Thus, in this embodiment, the candidate agent should both bind to Kif15 (although this may not be necessary), and alter its biological or biochemical activity as defined herein. The methods include both in vitro screening methods and in vivo screening of cells for alterations in cell cycle distribution, cell viability, or for the presence, morpohology, activity, distribution, or amount of mitotic spindles, as are generally outlined above.

Alternatively, differential screening may be used to identify drug candidates that bind to the native Kif15, but cannot bind to modified Kif15.

Positive controls and negative controls may be used in the assays. Preferably all control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g., albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

Administration

Accordingly, the compounds of the invention are administered to cells. By “administered” herein is meant administration of a therapeutically effective dose of a compound of the invention to a cell either in cell culture or in a patient. By “therapeutically effective dose” herein is meant a dose that produces the effects for which it is administered. The exact dose will depend on the purpose of the treatment, and will be ascertainable by one skilled in the art using known techniques. As is known in the art, adjustments for systemic versus localized delivery, age, body weight, general health, sex, diet, time of administration, drug interaction and the severity of the condition may be necessary, and will be ascertainable with routine experimentation by those skilled in the art. By “cells” herein is meant any cell in which mitosis or meiosis can be altered.

A “patient” for the purposes of the present invention includes both humans and other animals, particularly mammals, and other organisms. Thus the methods are applicable to both human therapy and veterinary applications. In the preferred embodiment the patient is a mammal, and in the most preferred embodiment the patient is human.

Compounds of the invention having the desired pharmacological activity may be administered, suitably as a pharmaceutically acceptable composition comprising an pharmaceutical excipient, to a patient, as described herein. Depending upon the manner of introduction, the compounds may be formulated in a variety of ways as discussed below. The concentration of therapeutically active compound in the formulation may vary from about 0.1-100 wt. %.

The agents may be administered alone or in combination with other treatments, i.e., radiation, or other chemotherapeutic agents such as the taxane class of agents that appear to act on microtubule formation or the camptothecin class of topoisomerase I inhibitors. When used, other chemotherapeutic agents may be administered before, concurrently, or after administration of a compound of the present invention. In one aspect of the invention, a compound of the present invention is co-administered with one or more other chemotherapeutic agents. By “co-administer” it is meant that the present compounds are administered to a patient such that the present compounds as well as the co-administered compound may be found in the patient's bloodstream at the same time, regardless when the compounds are actually administered, including simultaneously.

The administration of the compounds and compositions of the present invention can be done in a variety of ways, including, but not limited to, orally, subcutaneously, intravenously, intranasally, transdermally, intraperitoneally, intramuscularly, intrapulmonary, vaginally, rectally, or intraocularly. In some instances, for example, in the treatment of inflammation, the compound or composition may be directly applied as a solution or spray.

Pharmaceutical dosage forms include a compound of formula I or a pharmaceutically acceptable salt or solvate thereof, and one or more pharmaceutical excipients. As is known in the art, pharmaceutical excipients are secondary ingredients which function to enable or enhance the delivery of a drug or medicine in a variety of dosage forms (e.g.: oral forms such as tablets, capsules, and liquids; topical forms such as dermal, opthalmic, and otic forms; suppositories; injectables; respiratory forms and the like). Pharmaceutical excipients include inert or inactive ingredients, synergists or chemicals that substantively contribute to the medicinal effects of the active ingredient. For example, pharmaceutical excipients may function to improve flow characteristics, product uniformity, stability, taste, or appearance, to ease handling and administration of dose, for convenience of use, or to control bioavailability. While pharmaceutical excipients are commonly described as being inert or inactive, it is appreciated in the art that there is a relationship between the properties of the pharmaceutical excipients and the dosage forms containing them.

Pharmaceutical excipients suitable for use as carriers or diluents are well known in the art, and may be used in a variety of formulations. See, e.g., Remington's Pharmaceutical Sciences, 18th Edition, A. R. Gennaro, Editor, Mack Publishing Company (1990); Remington: The Science and Practice of Pharmacy, 20th Edition, A. R. Gennaro, Editor, Lippincott Williams & Wilkins (2000); Handbook of Pharmaceutical Excipients, 3rd Edition, A. H. Kibbe, Editor, American Pharmaceutical Association, and Pharmaceutical Press (2000); and Handbook of Pharmaceutical Additives, compiled by Michael and Irene Ash, Gower (1995), each of which is incorporated herein by reference for all purposes.

Oral solid dosage forms such as tablets will typically comprise one or more pharmaceutical excipients, which may for example help impart satisfactory processing and compression characteristics, or provide additional desirable physical characteristics to the tablet. Such pharmaceutical excipients may be selected from diluents, binders, glidants, lubricants, disintegrants, colors, flavors, sweetening agents, polymers, waxes or other solubility-retarding materials.

Compositions for intravenous administration will generally comprise intravenous fluids, i.e., sterile solutions of simple chemicals such as sugars, amino acids or electrolytes, which can be easily carried by the circulatory system and assimilated. Such fluids are prepared with water for injection USP.

Fluids used commonly for intravenous (IV) use are disclosed in Remington, the Science and Practice of Pharmacy [full citation previously provided], and include:

alcohol (e.g., in dextrose and water (“D/W”) [e.g., 5% dextrose] or dextrose and water [e.g., 5% dextrose] in normal saline solution (“NSS”); e.g. 5% alcohol);

synthetic amino acid such as Aminosyn, FreAmine, Travasol, e.g., 3.5 or 7; 8.5; 3.5, 5.5 or 8.5% respectively;

ammonium chloride e.g., 2.14%;

dextran 40, in NSS e.g., 10% or in D5/W e.g., 10%;

dextran 70, in NSS e.g., 6% or in D5/W e.g., 6%;

dextrose (glucose, D5/W) e.g., 2.5-50%;

dextrose and sodium chloride e.g., 5-20% dextrose and 0.22-0.9% NaCl;

lactated Ringer's (Hartmann's) e.g., NaCl 0.6%, KCl 0.03%, CaCl₂ 0.02%;

lactate 0.3%;

mannitol e.g., 5%, optionally in combination with dextrose e.g., 10% or NaCl e.g., 15 or 20%;

multiple electrolyte solutions with varying combinations of electrolytes, dextrose, fructose, invert sugar Ringer's e.g., NaCl 0.86%, KCl 0.03%, CaCl₂ 0.033%;

sodium bicarbonate e.g., 5%;

sodium chloride e.g., 0.45, 0.9, 3, or 5%;

sodium lactate e.g., ⅙ M; and

sterile water for injection

The pH of such fluids may vary, and will typically be from 3.5 to 8 such as known in the art.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All publications, including but not limited to patents and patent applications, cited in this specification are herein incorporated by reference as if each individual publication were specifically and individually indicated to be incorporated by reference herein as though fully set forth.

Experimental

The following is provided for exemplary synthesis of quinazolinediones of the invention, it is not meant to limit the scope of the invention in any way. Each reaction scheme is followed by an exemplary experimental procedure for making the specific compound illustrated. Each of the schemes and associated experimental procedures are chosen for illustrative purposes in order for the reader to fully understand the invention.

### EXAMPLE 1

Quinazolinedione Synthesis: Method A

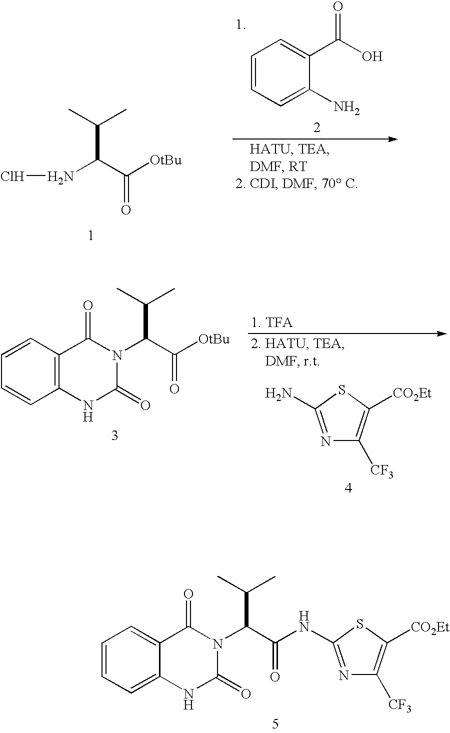

A solution of the Valine t-butyl ester hydrochloride (1, 7.6 g, 36.5 mmol), anthranilic acid (2, 5.0 g, 36.5 mmol), HATU (16.7 g, 44.0 mmol), TEA (20.3 mL, 146 mmol), and DMF (185 mL) was maintained at 23° C. for 6 hours. The reaction mixture was diluted with EtOAc (500 mL) and washed with saturated aqueous NH₄Cl (200 mL), saturated aqueous NaHCO₃ (200 mL), and brine (2×300 mL). The organic layer was dried (MgSO₄), filtered, and concentrated to provide a slightly yellow oil which was used without further purification.

The above crude amide (11.0 g, 36.5 mmol), carbonyldiimidazole (17.8 g, 109.5 mmol), and DMF (200 mL) was maintained at 70° C. for 2 hours. The reaction mixture was cooled to r.t., diluted with EtOAc (500 mL) and washed with saturated aqueous NH₄Cl (200 mL), saturated aqueous NaHCO₃ (200 mL), and brine (3×200 mL). The organic layer was dried (MgSO₄), filtered, and concentrated. The resulting residue was purified by flash column chromatography (5:1 hexanes:EtOAc; 4:1 hexanes:EtOAc; 3:1 hexanes:EtOAc; 2:1 hexanes:EtOAc) to yield 10.0 g (86%) of 3. LRMS (MH-tBu) m/z 263.1.

Quinazolinedione 3 (121 mg, 0.38 mmol) and TFA:H₂O (97.5:2.5, 2 mL) were maintained at 23° C. for 1 h. The reaction mixture was concentrated. The crude residue was diluted with EtOAc (20 mL) and washed with brine (10 mL). The organic layer was dried (MgSO₄), filtered, and concentrated to provide a white solid, which was used without further purification.

A solution of the above crude acid (100 mg, 0.38 mmol), ethyl 2-amino-4-trifluoromethylthiazole-5-carboxylate (4, 69 mg, 0.29 mmol), HATU (174 mg, 0.46 mmol), TEA (0.16 mL, 1.14 mmol), and DMF (2 mL) was maintained at 23° C. for 6 hours. The reaction mixture was diluted with EtOAc (20 mL) and washed with saturated aqueous NH₄Cl (10 mL), saturated aqueous NaHCO₃ (10 mL), and brine (2×10 mL). The organic layer was dried (MgSO₄), filtered, and concentrated. The resulting residue was purified by flash column chromatography (3:1 hexanes:EtOAc; 2:1 hexanes:EtOAc; 1:1 hexanes:EtOAc) to yield 68 mg (50%) of 5. LRMS (MH) m/z 485.0.

### EXAMPLE 2

Quinazolinedione Synthesis: Method B

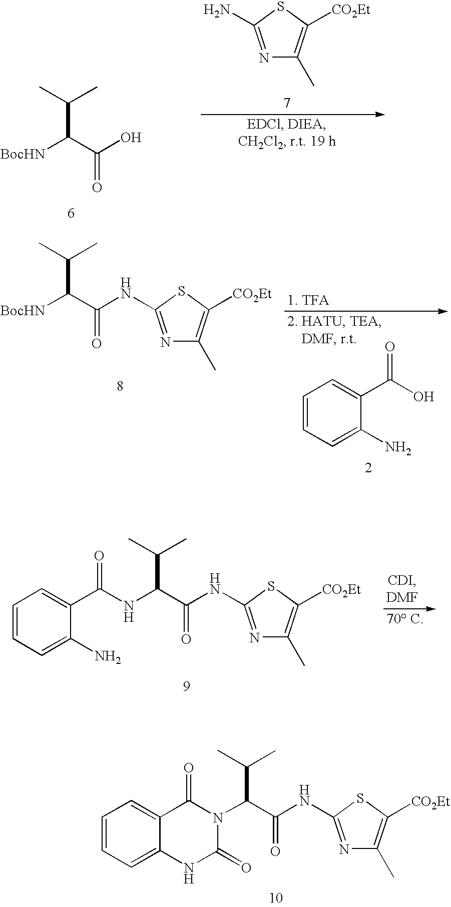

A mixture of Boc-Valine (6, 7.5 g, 34.5 mmol), ethyl 2-amino-4-methylthiazole-5-carboxylate (7, 6.5 g, 34.9 mmol), EDCI (8.0 g, 41.7 mmol), DIEA (22 mL, 126 mmol), DMAP (600 mg, 4.9 mmol), and CH₂Cl₂ (70 mL) were maintained at 23° C. for 19 hours. The reaction mixture was diluted with EtOAc (500 mL) and washed with saturated aqueous NH₄Cl (2×100 mL), 0.5 N NaOH (100 mL), and brine (100 mL). The organic layer was dried (MgSO₄), filtered, and concentrated. The resulting residue was purified by flash column chromatography (3:1 hexanes:EtOAc) to yield 11.0 g (83%) of 8. LRMS (MH) m/z 386.1.

Amide 8 (11.0 g, 28.5 mmol) and TFA:H₂O (97.5:2.5, 60 mL) was maintained at 23° C. for 1 h. The reaction mixture was concentrated to provide a colorless oil, which was used without further purification.

A solution of a portion of the above crude amine (3.99 g, 10.0 mmol), anthranilic acid (2, 1.37 g, 10.0 mmol), HATU (4.18 g, 11.0 mmol), TEA (4.1 mL, 30.0 mmol), and DMF (40 mL) was maintained at 23° C. for 6 hours. The reaction mixture was diluted with EtOAc (200 mL) and washed with saturated aqueous NH₄Cl (100 mL), saturated aqueous NaHCO₃ (100 mL), and brine (2×100 mL). The organic layer was dried (MgSO₄), filtered, and concentrated. The resulting residue was purified by flash column chromatography (3:1 hexanes:EtOAc; 2:1 hexanes:EtOAc; 1:1 hexanes:EtOAc) to yield 3.13 g (77%) of 9. LRMS (MH) m/z 405.1.

A solution of amide 9 (3.13 g, 7.75 mmol), carbonyldiimidazole (3.75 g, 23.2 mmol), and DMF (50 mL) was maintained at 70° C. for 1 hour. The reaction mixture was cooled to r.t., diluted with EtOAc (200 mL) and washed with saturated aqueous NH₄Cl (100 mL), saturated aqueous NaHCO₃ (100 mL), and brine (2×100 mL). The organic layer was dried (MgSO₄), filtered, and concentrated. The resulting residue was purified by flash column chromatography (3:1 hexanes:EtOAc; 2:1 hexanes:EtOAc; 1:1 hexanes:EtOAc) to yield 2.67 g (80%) of 6. LRMS (MH) m/z 431.1.

### EXAMPLE 3

Synthesis of Oxadiazoles.

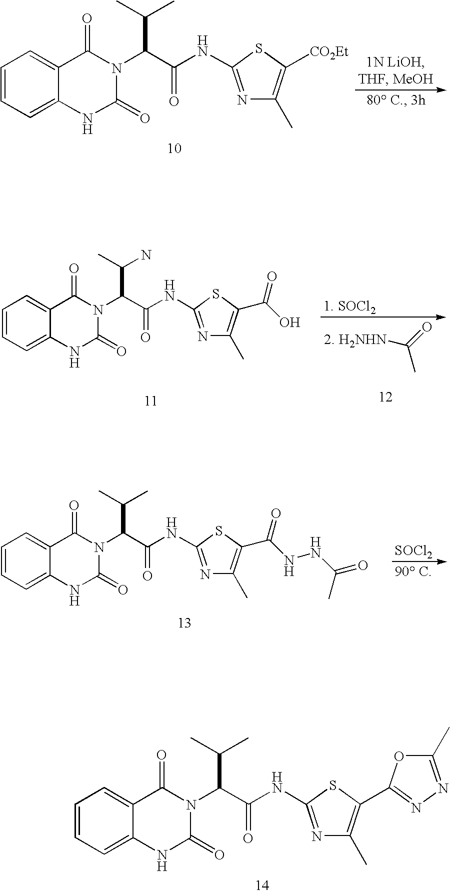

A solution of quinazolinedione 10 (1.50 g, 34.9 mmol), 1N LiOH (30 mL), THF (20 mL), and MeOH (6 mL) was maintained at 80° C. for 3 hours. The reaction mixture was cooled to r.t., quenched with 1N HCl (70 mL), and extracted with EtOAc (3×200 mL) and CHCl₃ (2×200 mL). The organic layers were dried (MgSO₄), filtered, and concentrated. The resulting white solid, 1.27 g (90%), was used without further purification LRMS (MH) m/z 403.1.

A solution of acid 11 (205 mg, 0.51 mmol), thionyl chloride (3 mL), and DMF (50 μL) was maintained at r.t. for 1 h. The reaction mixture was then concentrated and placed under vacuum (0.1 mmHg) for 2 hours. The resulting oil was used without further purification.

To a r.t. solution of the above acid chloride (˜0.51 mmol) and acetic acid hydrazide (12, 150 mg, 2.0 mmol), and CH₂Cl₂ (10 mL) was added TEA (0.4 mL, 2.9 mmol). After 30 mins, the reaction mixture was diluted with EtOAc (20 mL) and washed with saturated aqueous NH₄Cl (10 mL), saturated aqueous NaHCO₃ (10 mL), and brine (10 mL). The organic layer was dried (MgSO₄), filtered, and concentrated. The resulting residue was used without further purification.

A solution of crude quinazolinedione 13 (˜0.51 mmol) and thionyl chloride (5 mL) was heated to 90° C. for 3 hours. The reaction mixture was concentrated and the crude residue was purified by flash column chromatography (3:1 hexanes:EtOAc; 2:1 hexanes:EtOAc; 1:1 hexanes:EtOAc) to yield 22 mg (10%) of 14. LRMS (MH) m/z 441.1.

### EXAMPLE 4

Synthesis of Oxadiazoles.

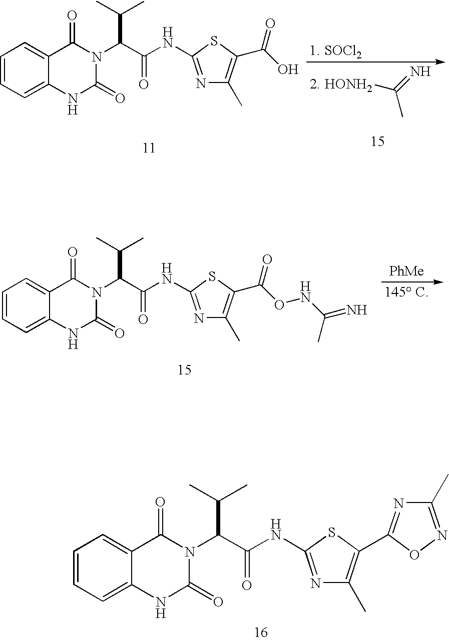

A solution of acid 11 (162 mg, 0.40 mmol), thionyl chloride (3 mL), and DMF (50 μL) was maintained at r.t. for 1 h. The reaction mixture was then concentrated and placed under vacuum (0.1 mmHg) for 2 hours. The resulting oil was used without further purification.

A solution of the above acid chloride (˜0.40 mmol), hydroxylamine acetamide (15, 85 mg, 2.0 mmol), CH₂Cl₂ (3 mL), and DMF (3 mL) was maintained at r.t for 30 mins. The reaction mixture was then diluted with EtOAc (20 mL) and washed with saturated aqueous NaHCO₃ (10 mL), and brine (10 mL). The organic layer was dried (MgSO₄), filtered, and concentrated. The resulting residue was used without further purification.

A mixture of crude quinazolinedione 15 (˜0.41 mmol) and toluene (5 mL) was heated to 145° C. in a sealed tube for 15 mins. The reaction mixture was concentrated and the crude residue was purified by flash column chromatography (3:1 hexanes:EtOAc; 2:1 hexanes:EtOAc; 1:1 hexanes:EtOAc) to yield 15 mg (8%) of 16. LRMS (MH) m/z 441.1.

### EXAMPLE 5

Synthesis of Oxadiazoles.

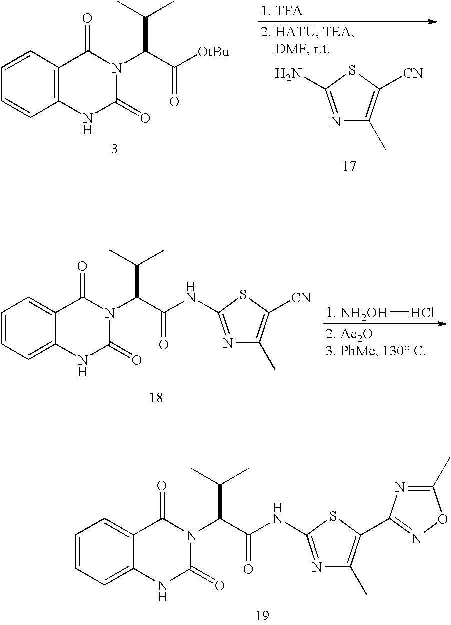

Quinazolinedione 3 (503 mg, 1.58 mmol) and TFA:H₂O (97.5:2.5, 10 mL) were maintained at 23° C. for 1 h. The reaction mixture was concentrated. The crude residue was diluted with EtOAc (40 mL) and washed with brine (10 mL). The organic layer was dried (MgSO₄), filtered, and concentrated to provide a white solid, which was used without further purification.

A solution of the above crude acid (415 mg, 1.58 mmol), ethyl 2-amino-4-cyanothiazole-5-carboxylate (17 (Murata, et. al. Bull. Chem. Soc. Jpn. 1952, 25, 16), 200 mg, 1.44 mmol), HATU (821 mg, 2.16 mmol), TEA (0.8 mL, 5.8 mmol), and DMF (4 mL) was maintained at 23° C. for 18 hours. The reaction mixture was diluted with EtOAc (20 mL) and washed with saturated aqueous NH₄Cl (10 mL), saturated aqueous NaHCO₃ (10 mL), and brine (2×10 mL). The organic layer was dried (MgSO₄), filtered, and concentrated. The resulting residue was purified by flash column chromatography (1:1 hexanes:EtOAc) to yield 350 mg (58%) of 18. LRMS (MH) m/z 383.1.

A mixture of quinazolinedione 18 (100 mg, 0.26 mmol), hydroxylamine hydrochloride (100 mg, 1.44 mmol), Na₂CO₃ (200 mg, 1.89 mmol), and EtOH (2 mL) was maintained at 70° C. for 1.5 hours. The reaction mixture was diluted with EtOAc (20 mL), filtered through a pad of Celite, and the filtrate was concentrated. The crude residue was used without further purification.

The crude quinazolinedione (˜0.26 mmol), acetic anyhydride (50 μL, 0.8 mmol), pyridine (50 μL, 0.6 mmol), and CH₂Cl₂ (3 mL) were maintained at r.t. for 3 hours. The reaction mixture was diluted with EtOAc (20 mL) and washed with saturated aqueous NaHCO₃ (10 mL), and brine (10 mL). The organic layer was dried (MgSO₄), filtered, and concentrated. The crude residue was used without further purification.

A mixture of the above crude quinazolinedione (˜0.26 mmol) and toluene (5 mL) was heated to 145° C. in a sealed tube for 15 mins. The reaction mixture was concentrated and the crude residue was purified by flash column chromatography (3:1 hexanes:EtOAc; 2:1 hexanes:EtOAc; 1:1 hexanes:EtOAc) to yield 18 mg (16%) of 19. LRMS (MH) m/z 441.1.

### EXAMPLE 6

Alkylation of quinazolinedione-3-nitrogen.

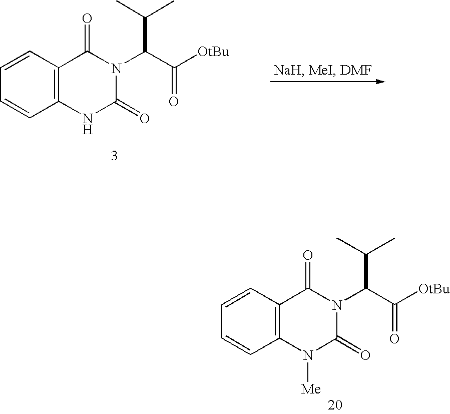

A solution of quinazolinedione 3 (540 mg, 1.42 mmol), sodium hydride (85 mg, 2.13 mmol), iodomethane (0.13 mL, 2.14 mmol), and DMF (5 mL) was maintained at r.t. for 1 hour. The reaction mixture was diluted with EtOAc (30 mL) and washed with saturated aqueous NH₄Cl (20 mL), saturated aqueous NaHCO₃ (20 mL), and brine (2×20 mL). The organic layer was dried (MgSO₄), filtered, and concentrated. The resulting residue was purified by flash column chromatography (3:1 hexanes:EtOAc) to yield 353 mg (75%) of 20.

### EXAMPLE 7

Introduction of R₅.

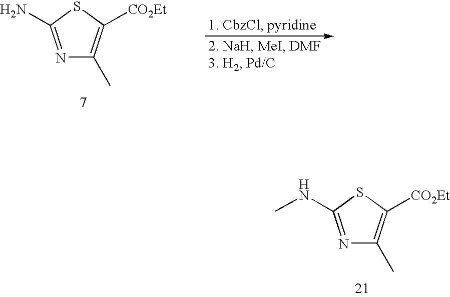

Benzyloxychloroformate (CbzCl, 3.92 mL, 27.4 mmol) was added to a r.t. solution of ethyl ²-amino-4-methylthiazole-5-carboxylate (7, 5.11 g, 27.4 mmol), pyridine (4.44 mL, 54.9 mmol), and CH₂Cl₂ (200 mL). After 1 hour, the reaction mixture was washed with saturated aqueous NH₄Cl (100 mL), saturated aqueous NaHCO₃ (100 mL), and brine (100 mL). The organic layer was dried (MgSO₄), filtered, and concentrated. The resulting white solid was used without further purification.

The above aminothiazole (300 mg, 0.94 mmol), sodium hydride (75 mg, 1.90 mmol), iodomethane (0.12 mL, 1.90 mmol), and DMF (5 mL) was maintained at r.t. for 1 hour. The reaction mixture was diluted with EtOAc (30 mL) and washed with saturated aqueous NH₄Cl (20 mL), saturated aqueous NaHCO₃ (20 mL), and brine (2×20 mL). The organic layer was dried (MgSO₄), filtered, and concentrated. The resulting residue was purified by flash column chromatography (5:1 hexanes:EtOAc) to yield 250 mg (80%) of product.

The above methylated aminothiazole (250 mg, 0.75 mmol), 10% Pd on carbon (100 mg), and EtOAc (15 mL) was hydrogenated (1 atm) for 3 h at r.t. The reaction mixture was then filtered through Celite and concentrated to provide 150 mg (100%) of 21.

### EXAMPLE 8

SKOV-3 Assay

Human tumor cells Skov-3 (ovarian) were plated in 96-well plates at densities of 4,000 cells per well, allowed to adhere for 24 hours, and treated with various concentrations of the Kif15 inhibitors described herein for 24 hours. Cells were fixed in 4% formaldehyde and stained with antitubulin antibodies (subsequently recognized using fluorescently-labeled secondary antibody) and Hoechst dye (which stains DNA). Visual inspection revealed that the compounds caused cell cycle arrest.

### EXAMPLE 9

Inhibition of Cellular Proliferation in Tumor Cell Lines

Cells were plated in 96-well plates at densities from 1000-2500 cells/well of a 96-well plate and allowed to adhere/grow for 24 hours. They were then treated with various concentrations of drug for 48 hours. The time at which compounds are added is considered T₀. A tetrazolium-based assay using the reagent 3-(4,5-dimethylthiazol-2-yl)-5-(3-carboxymethoxyphenyl)-2-(4-sulfophenyl)-2H-tetrazolium (MTS) (I.S>U.S. Pat. No. 5,185,450) (see Promega product catalog HG3580, CellTiter 96® AQᵤₑₒᵤₛ One Solution Cell Proliferation Assay) was used to determine the number of viable cells at T₀ and the number of cells remaining after 48 hours compound exposure. The number of cells remaining after 48 hours was compared to the number of viable cells at the time of drug addition, allowing for calculation of growth inhibition.

The growth over 48 hours of cells in control wells that had been treated with vehicle only (0.25% DMSO) is considered 100% growth and the growth of cells in wells with compounds is compared to this. Kif15 inhibitors inhibited cell proliferation in human ovarian tumor cell lines (SKOV-3).

A Gi₅₀ was calculated by plotting the concentration of compound in μM vs the percentage of cell growth of cell growth in treated wells. The Gi₅₀ calculated for the compounds is the estimated concentration at which growth is inhibited by 50% compared to control, i.e., the concentration at which:

100×[(Treated₄₈−T₀)/(Control₄₈−T₀)]=50.

All concentrations of compounds are tested in duplicate and controls are averaged over 12 wells. A very similar 96-well plate layout and Gi₅₀ calculation scheme is used by the National Cancer Institute (see Monks, et al., J. NatI. Cancer Inst. 83:757-766 (1991)). However, the method by which the National Cancer Institute quantitates cell number does not use MTS, but instead employs alternative methods.

### EXAMPLE 10

Calculation of IC₅₀:

Measurement of a compound's IC₅₀ for Kif15 activity uses an ATPase assay. The following solutions are used: Solution 1 consists of 3 mM phosphoenolpyruvate potassium salt (Sigma P-7127), 2 mM ATP (Sigma A-3377), 1 mM IDTT (Sigma D-9779), 5 μM paclitaxel (Sigma T-7402), 10 ppm antifoam 289 (Sigma A-8436), 25 mM Pipes/KOH pH 6.8 (Sigma P6757), 2 mM MgC12 (VWR JT400301), and 1 mM EGTA (Sigma E3889). Solution 2 consists of 1 mM NADH (Sigma N8129), 0.2 mg/ml BSA (Sigma A7906), pyruvate kinase 7 U/ml, L-lactate dehydrogenase 10 U/ml (Sigma P0294), 100 nM Kif15 motor domain, 50 μg/ml microtubules, 1 mM DTT (Sigma D9779), 5 μM paclitaxel (Sigma T-7402), 10 ppm antifoam 289 (Sigma A-8436), 25 mM Pipes/KOH pH 6.8 (Sigma P6757), 2 mM MgC12 (VWR JT4003-01), and 1 mM EGTA (Sigma E3889). Serial dilutions (8-12 two-fold dilutions) of the compound are made in a 96-well microtiter plate (Corning Costar 3695) using Solution 1. Following serial dilution each well has 50 μl of Solution 1. The reaction is started by adding 50 μl of solution 2 to each well. This may be done with a multichannel pipettor either manually or with automated liquid handling devices. The microtiter plate is then transferred to a microplate absorbance reader and multiple absorbance readings at 340 nm are taken for each well in a kinetic mode. The observed rate of change, which is proportional to the ATPase rate, is then plotted as a function of the compound concentration. For a standard IC₅₀ determination the data acquired is fit by the following four parameter equation using a nonlinear fitting program (e.g., Grafit 4):

$$y = \frac{Range}{1 + {( \frac{x}{I {C}_{50}} )}^{s}} + Background$$

where y is the observed rate and x the compound concentration.

Other compounds of this class were found to inhibit cell proliferation, although GI₅₀ values varied. Many of the compounds have GI₅₀ values less than 10 μM, and several have GI₅₀ values less than 1 μM. Anti-proliferative compounds that have been successfully applied in the clinic to treatment of cancer (cancer chemotherapeutics) have GI₅₀'s that vary greatly. For example, in A549 cells, paclitaxel GI₅₀ is 4 nM, doxorubicin is 63 nM, 5-fluorouracil is 1 μM, and hydroxyurea is 500 μM (data provided by National Cancer Institute, Developmental Therapeutic Program, http://dtp.nci.nih.gov/). Therefore, compounds that inhibit cellular proliferation at virtually any concentration may be useful. However, preferably, compounds will have GI₅₀ values of less than 1 mM. More preferably, compounds will have GI₅₀ values of less than 20 μM. Even more preferably, compounds will have GI₅₀ values of less than 10 μM. Further reduction in GI₅₀ values may also be desirable, including compounds with GI₅₀ values of less than 1 μM. Some of the compounds of the invention inhibit cell proliferation with GI₅₀ values from below 200 nM to below 10 nM.

What is claimed is:

1. A compound having the structure: 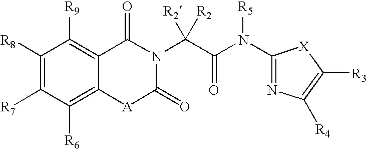
wherein
A is a bond or is —NR₁— wherein R₁ is hydrogen, alkyl, or substituted alkyl;
X is O, S, or —NR₁₂— wherein R₁₂ is hydrogen, alkyl, or substituted alkyl;
R₂ and R₂′ are independently selected from the group consisting of hydrogen, optionally substituted alkyl, optionally substituted aryl, optionally substituted aralkyl, optionally substituted heterocyclyl, and optionally substituted heterocyclylalkyl, or R₂ and R₂′ taken together form an optionally substituted 3- to 7-membered ring;
R₃ is carboxy, alkoxycarbonyl, optionally substituted lower-alkyl, or optionally substituted heterocyclyl;
R₄ is hydrogen or optionally substituted lower-alkyl;
R₅ is hydrogen or optionally substituted lower-alkyl; and
R₆, R₇, R₈, and R₉ are independently selected from the group consisting of hydrogen, optionally substituted alkyl, optionally substituted alkoxy, alkoxycarbonyl, halogen, hydroxy, cyano, nitro, amino, alkylamino, dialkylamino, optionally substituted alkylsulfanyl, optionally substituted alkylsulfonyl, optionally substituted alkylsulfonamido, optionally substituted arylsulfonamido, carboxamido, aminocarbonyl, optionally substituted aryl, and optionally substituted heterocyclyl;
including single stereoisomers, mixtures of stereoisomers, and the pharmaceutically acceptable salts thereof.

2. The compound of claim 1, wherein A is —NR₁; R₁ is hydrogen, alkyl, or substituted alkyl, and X is S.

3. The compound of claim 2, wherein R₁ is hydrogen.

4. The compound of claim 1, wherein R₂ and R₂′ are independently selected from the group consisting of hydrogen, optionally substituted alkyl, optionally substituted aryl, optionally substituted aralkyl, optionally substituted heterocyclyl, optionally substituted heterocyclylalkyl, or R₂ and R₂′ taken together form an optionally substituted 3- to 7-membered ring.

5. The compound of claim 4, wherein R₂′ is hydrogen.

6. The compound of claim 4 or 5, wherein R₂ is phenyl, lower-alkyl or substituted lower-alkyl.

7. The compound of claim 6, wherein R₂ is phenyl, methyl, ethyl, i-propyl, n-propyl, i-butyl, s-butyl, or n-propyl.

8. The compound of claim 6, wherein the stereogenic center to which R₂ and R₂′ are attached is of the S-configuration.

9. The compound of claim 1, wherein R₃ is carboxy, alkoxycarbonyl, optionally substituted lower-alkyl, or optionally substituted heterocyclyl.

10. The compound of claim 9, wherein R₃ is carboxy, alkoxycarbonyl, or optionally substituted oxadiazyl.

11. The compound of claim 10, wherein R₃ is —(CO)OR₁₀ wherein R₁₀ is lower-alkyl.

12. The compound of claim 11, wherein R₁₀ is methyl, ethyl, or propyl.

13. The compound of claim 10, wherein R₃ is 3-R₁₁-1,2,4-oxadiazyl, 5-R₁₁-1,2,4-oxadiazyl, or 5-R₁₁-1,3,4-oxadiazyl wherein R₁₁ is lower-alkyl.

14. The compound of claim 1, wherein R₄ is hydrogen or optionally substituted lower-alkyl.

15. The compound of claim 14, wherein R₄ is lower-alkyl or substituted lower-alkyl.

16. The compound of claim 15, wherein R₄ is methyl or trifluoromethyl.

17. The compound of claim 1, wherein R₅ is hydrogen or optionally substituted lower-alkyl.

18. The compound of claim 17, wherein R₅ is hydrogen or methyl.

19. The compound of claim 1, wherein R₆, R₇, R₈, and R₉ are independently selected from the group consisting of R₆, R₇, R₈, and R₉ are independently selected from the group consisting of hydrogen, optionally substituted alkyl, optionally substituted alkoxy, alkoxycarbonyl, halogen, hydroxy, cyano, nitro, amino, alkylamino, dialkylamino, optionally substituted alkylsulfanyl, optionally substituted alkylsulfonyl, optionally substituted alkylsulfonamido, optionally substituted arylsulfonamido, carboxamido, aminocarbonyl, optionally substituted aryl, and optionally substituted heterocyclyl.

20. The compound of claim 19, wherein R₆, R₇, R₈, and R₉ are independently selected from the group consisting of hydrogen, halogen, hydroxy, lower-alkyl, substituted lower-alkyl, and lower-alkoxy.

21. The compound of claim 1, wherein
A is —NR₁;
R₁ is hydrogen, alkyl, or lower-alkyl;
X is S;
R₂′ is hydrogen;
R₂ is optionally substituted lower-alkyl;
R₃ is alkoxycarbonyl or optionally substituted oxadiazyl;
R₄ is hydrogen or optionally substituted lower-alkyl;
R₅ is hydrogen or optionally substituted lower-alkyl; and
R₆, R₇, R₈, and R₉ are independently selected from the group consisting of hydrogen, halogen, hydroxy, optionally substituted lower-alkyl, and optionally substituted alkoxy.

22. A composition comprising a pharmaceutically acceptable excipient and the compound or salt thereof of any one of claims 1-21.

23. A composition according to claim 22, wherein said composition further comprises a chemotherapeutic agent.

24. A composition according to claim 23, wherein said composition further comprises a taxane.

25. A composition according to claim 23, wherein said composition further comprises a vinca alkaloid.

26. A composition according to claim 23, wherein said composition further comprises a topoisomerase I inhibitor.

27. A method of inhibiting Kif15 kinesin activity which comprises contacting said kinesin with an effective amount of the compound according to any one of claims 1 to 21, or a pharmaceutically acceptable salt thereof.

28. A method of inhibiting Kif15 which comprises contacting said kinesin with an effective amount of the compound according to any one of claims 1 to 21, or a pharmaceutically acceptable salt thereof.

29. A method for the treatment of a disease of proliferating cells comprising administering to a subject in need thereof the compound according to any one of claims 1-21, or a pharmaceutically acceptable salt thereof.

30. A method for the treatment of a disease of proliferating cells comprising administering to a subject in need thereof the composition according to any one of claims 22-26.

31. A method according to claim 29 or claim 30 wherein said disease is selected from the group cancer, hyperplasias, restenosis, cardiac hypertrophy, immune disorders, and inflammation.

32. The use, in the manufacture of a medicament for treating cellular proliferative disease, of a compound according to any one of claims 1-21, or a pharmaceutically acceptable salt thereof.

33. The use of a compound as defined in claim 32 for the manufacture of a medicament for treating a disorder associated with Kif15 kinesin activity.
